(19) United States
(12) Patent Application Publication
(10) Pub. No.: US 2024/0003903 A1
(43) Pub. Date: Jan. 4, 2024

(54) SYSTEMS AND REAGENTS FOR DETECTION OF FREE MONOCLONAL IMMUNOGLOBULIN LIGHT CHAINS IN BIOLOGICAL SAMPLES

(71) Applicant: Augusta University Research Institute, Inc., Augusta, GA (US)

(72) Inventors: Gurmukh Singh, Augusta, GA (US); Roni J. Bollag, Martinez, GA (US)

(21) Appl. No.: 18/346,483

(22) Filed: Jul. 3, 2023

Related U.S. Application Data
(60) Provisional application No. 63/357,801, filed on Jul. 1, 2022.

(51) Int. Cl. G01N 33/68; G01N 33/574; C07K 16/42

(52) U.S. Cl. CPC G01N 33/6857; G01N 33/57407; C07K 16/42; G01N 2800/52

(57) ABSTRACT

Compositions and methods for determining the presence of free monoclonal immunoglobulin light chains in biological samples with improved resolution and sensitivity are described. The methods detect subjects who have or are at risk of neoplastic monoclonal gammopathies and identify residual/minimal residual disease in subjects who have received therapy for neoplastic monoclonal gammopathies. The methods include immunofixation electrophoresis modified by applying undiluted or concentrated biological samples, washing/blotting of gels to enhance removal of residual proteins, and staining for free light chains with antisera specific to free light chains. Kits including compositions required for the methods are also provided.

### CROSS-REFERENCE TO RELATED APPLICATIONS

This application claims the benefit of and priority to U.S. Provisional Application No. 63/357,801 filed Jul. 1, 2022, which is hereby incorporated by reference in its entirety.

### FIELD OF THE INVENTION

The invention is generally related to systems and reagents for high-resolution detection of gammopathies, particularly screening of serum, urine other body fluids, cell and tissue extracts and bone marrow samples for diagnosis of monoclonal gammopathy disorders and residual and minimal residual diseases in patients undergoing treatment for multiple myeloma.

### BACKGROUND OF THE INVENTION

Neoplastic monoclonal gammopathies (NMG) include monoclonal gammopathy of undetermined significance (MGUS), asymptomatic or smoldering multiple myeloma (SMM) and multiple/plasma cell myeloma (MM) (Kyle, et al., N. Engl. J. Med. 2018; 378:241-249; Lakshman, et al., Blood Cancer J. 2018; 12(8):59-69; Palumbo, et al., N. Engl. J. Med. 2011; 364:1046-1060). The diagnostic criteria for these entities are well described and generally accepted (Singh, J. Appl. Lab. Med. 2020; 5:1358-1371). Of these only the malignant entity, MM, is treated in routine clinical care with antineoplastic drugs. By contrast the pre-malignant conditions of MGUS and SMM are usually observed, and treatment initiated when the lesions meet the criteria for MM (Fonseca, et al. Am. Soc. Clin. Oncol. Educ. Book. 2020; 40:1-7; Lonial, et al., J. Clin. Oncol. 2020; 38:1126-1137; Kim, et al., Cancers. 2020; 12:1223-1240).

Multiple myeloma is a malignant tumor of plasma cells and is generally associated with synthesis and secretion of monoclonal immunoglobulins by tumor cells. MM is the second commonest hematologic malignancy in adults and accounts for about 2% of cancer deaths. The tumor is treatable but incurable. Improvements in drug treatment and autologous stem cell transplantation (ASCT) have improved survival such that survival beyond ten years is not uncommon (Kazandjian, Semin. Oncol. 2016; 43:676-681; Dhakal, et al., JAMA Oncol. 2018; 4:343-350; Attal, et al., N. Engl. J. Med. 2017; 376:1311-1320; Jin, et al., J. Appl. Lab. Med. 2021 Sep. 1 doi: 10.1093/jalm/jfab090).

Staging systems for myeloma, including the Durie-Salmon and International System take into account clinical and laboratory parameters of the extent of disease in addressing prognosis. Factors inherent to the tumor that portend poorer outcomes are the presence of del (17p) and/or translocation t(4;14), t(14;16), t(14;20), and amplification of 1q21. Partial or complete deletion of chromosome 13, 17p13 deletion and deletion 1p are additional markers of adverse outcome. The plasma cell labelling index (PCLI) may predict time to disease progression and death though currently PCLI is rarely used because of the availability of more practical prognostic methods (Sonneveld, et al., International Myeloma Working Group. Blood. 2016; 127:2955-2962).

Higher levels of serum free monoclonal light chains (SFLC) have been observed to result in shorter survival, perhaps through induction of renal damage. In MM lesions secreting intact immunoglobulins, a sub-group of about 18% of the tumors produce marked excess of free monoclonal light chains. These light-chain-predominant MM (LCPMM) have significantly lower eGFR and shorter survival. On analyzing SFLC and intact monoclonal immunoglobulin levels, the inflection/change point for identifying this subgroup of LCPMM was observed to be at 67 mg/L of SFLC per gram/dL of intact immunoglobulins for kappa light chain associated lesions. The corresponding value for lambda light chain associated lesions was 43.5 mg/L/g of monoclonal immunoglobulin (Singh, et al., Lab. Med. 2020 Nov. 12 doi:10.1093/labmed/lmaa057).

About 15% of MM lesions secrete light chains only, i.e., light chain multiple myelomas (LCMM). Within this group of LCMM about 40% of the lesions have markedly higher levels of SFLC. The inflection/change point for separating high level of SFLC was observed to be at 455 mg/L. Due to the smaller number of patients observed, separate inflection/change points for kappa and lambda lesions were not calculated. As in the case of LCPMM, the high SFLC subgroup of LCMM exhibited significantly lower eGFR and significantly shorter survival. No specific, effective treatments are available for addressing high monoclonal free light chains. Use of plasmapheresis and dialysis with a larger pore membrane have not shown consistent beneficial results (Manohar, et al., Curr. Hematol. Malig. Rep. 2018; 13:220-226).

The diagnostic work-up for MM includes multiple laboratory tests. The tests recommended by the International Myeloma Working Group (IMWG) in the diagnosis and monitoring of monoclonal gammopathy disorders include complete blood cell count (CBC), comprehensive metabolic prolife (CMP), immunoglobulin quantification, serum free light chain (SFLC) concentration, serum protein electrophoresis (SPEP) and serum protein immunofixation electrophoresis (SIFE), urine protein electrophoresis (UPEP) and urine protein immunofixation electrophoresis (UIFE). UPEP per se is not useful for detection of monoclonal immunoglobulins in urine, but has traditionally been performed along with UIFE. UPEP may provide useful information about kidney disorders, but it does not add value in the diagnosis and follow-up of patients with monoclonal gammopathy. Traditionally, gel or capillary based electrophoretic methods have been used for detection, quantification, and monitoring of monoclonal immunoglobulins. Gel-based methods employ serum protein electrophoresis (SPEP) and serum immunofixation electrophoresis (SIFE) and are standard laboratory tests at most medical centers. Capillary zonal electrophoresis (CE) and immunosubtraction electrophoresis (ISUB) are equivalent techniques using a more automated capillary fluidic electrophoresis method. The concentration of monoclonal immunoglobulins (MIg) in neoplastic monoclonal gammopathies is generally measured by densitometric scanning of monoclonal peaks on gel electrophoresis, or by the measured peak area guided by immunosubtraction (ISUB) on CE. Quantification by these two methods produce comparable results (Keren, et al., Clin. Chem. Lab. Med. 2016, 54:947-961; Omar, et al., Lab. Med. 2021 Aug. 13 doi: 10.1093/labmed/lmab055. lmab055. doi: Online ahead of print).

A major innovation has been the introduction of an assay for assessing free light chains in serum, initially described by Bradwell, et al., in Serum Free Light Chain Analysis Plus Hevylite. seventh ed., ISBN 780-0-9932196-0-3. Based on the molecular mechanisms driving immunoglobulin gene rearrangement during the synthesis of immunoglobulins, plasma cells tend to produce more light chains than heavy chains. This excess production of light chains extends to polyclonal gammopathy and neoplastic monoclonal gammopathy (NMG). In neoplastic disorders of plasma cells, serum and urine contain free monoclonal light chains and these can serve as diagnostic and monitoring tools for NMG (Lee and Singh, Lab. Med. 2019, 50:381-389; Singh, Lab. Med. 2019, 50(2):189-193; Lee and Singh, J. Clin. Med. Res. 2018; 10:562-569). In LCMM and LCPMM, measuring and monitoring of SFLC provides a practical method for monitoring the course of disease. The proposed role for enumerating the ratio of kappa to lambda SFLC has not proven to be particularly useful due to a high incidence of false positive, false negative, and incongruent results especially following autologous stem cell transplantation (Singh, Am. J. Clin. Pathol. 2016, 146:207-214; Singh, J. Clin. Med. Res. 2017, 9:46-57; Singh, J. Clin. Med. Res. 2017, 9:671-679.). In general, the NMG produce more kappa chains than lambda chains and this disparity complicates the results of Kappa/Lambda ratio and involved/uninvolved light chain ratio especially following hematopoietic stem cell transplants.

Recent advances in detecting monoclonal immunoglobulins in general, and monoclonal light chains in particular, include the use of mass-spectrometry. Nanobody-mediated concentration of immunoglobulins followed by matrix desorption time of flight analysis (MALDI-TOF) has been described as a screening tool for monoclonal immunoglobulins and presented as an assay with higher sensitivity than conventional methods. It has been promoted for detection of minimal residual disease (MRD) (Sepiashvili, et al., Clin. Chem. 2019, 65:1015-1022; Mills, et al., Clin. Chem. 2016, 62:1334-1344; Zajec, et al., Clin. Chem. 2020, 66:421-433; Milani, et al., Am. J. Hematol. 2017, 92:772-779). However, current systems for quantitation of monoclonal immunoglobulins are reliant on densitometric scanning of SPEP or with CE, by using ISUB to guide demarcation.

Neoplastic gammopathies that predominantly (or exclusively) produce free light chains can be hard to detect, especially when looking for residual or minimal residual disease in the post-treatment setting. The current standard of practice is overly reliant on the serum free light chain assay, which is fraught with false positives, false negatives, and an intrinsic inability to differentiate monoclonal from polyclonal light chains.

Standard IFEs frequently fail to detect monoclonal free light chain bands due to a number of factors, including (a) over-dilution of serum per standard protocols, (b) comigration of the monoclonal light chain bands and intact monoclonal immunoglobulins precludes distinction of light chains from the intact immunoglobulin band, (c) and possibly due to relatively poor binding affinity between conventional anti-kappa and anti-lambda antisera and monoclonal free light chains compared to standard antisera's affinity for light chains complexed to heavy chains.

Therefore, there is a need for improved systems and reagents for the robust, reproducible, high-resolution detection and quantitation of free monoclonal light chain proteins within biological samples such as serum urine, other body fluids, lysates of cells and tissues and lysate of bone marrow cells, to facilitate diagnosis and monitoring of neoplastic monoclonal gammopathies (NMG)

Thus, it is an object of the invention to provide systems and reagents for the improved detection and measurement of molecular determinants of gammopathies, including free monoclonal light chains.

It is another object of the invention to provide improved systems and reagents to inform the status and/or monitor the progression of neoplastic monoclonal gammopathies (NMG).

It is another object of the invention to provide improved systems and reagents to assess the outcome of therapies for neoplastic monoclonal gammopathies (NMG).

It is yet another object of the invention to provide systems and reagents for rapid, high-sensitivity detection of monoclonal light chains within serum, urine, other body fluids and cell and tissue lysates to identify and inform treatment regimens for MM, residual and minimal residual disease in patients undergoing treatment for neoplastic monoclonal gammopathies (NMG).

### SUMMARY OF THE INVENTION

Enhanced methods for the detection and quantitation of free immunoglobulin chains in a biological sample have been developed. Any level of free monoclonal light chains in a biological fluid is abnormal and portends NMG or risk of NMG. Monoclonal light chains are distinguished from polyclonal free light chains, for example, in stained FLC-Modified SIFE gels by recognizing the different staining patterns generated by monoclonal vs, polyclonal free light chains. Compositions and methods for high-resolution quantitation of free monoclonal immunoglobulin light chain proteins within serum, urine, other body fluids, and cell and tissue lysates have been developed.

Methods of detecting serum free monoclonal light chains are provided. Methods of identifying a subject as having a disease or disorder associated with serum free monoclonal light chains, or as being at risk of having a disease or disorder associated with serum free monoclonal light chains are also provided and can be used in conjunction with the methods of detection. Any of the methods can include separating proteins within a biological sample, preferably an undiluted biological sample, from the subject to create a protein separation profile (e.g., undiluted protein separation profile), selectively labeling free immunoglobulin light chain proteins within the protein profile (e.g., undiluted protein profile), and quantifying the labelled free monoclonal immunoglobulin light chain proteins. The presence of more than about 1.75 mg/L free monoclonal immunoglobulin light chain in serum, urine or other specimens is an indication that a subject has or is at risk of a disease or disorder associated with serum free monoclonal light chains, such as multiple myeloma and in post-treatment persons with residual/minimal residual disease (MRD) of multiple myeloma.

Typically, methods of detecting free monoclonal light chains include immunofixation electrophoresis (SIFE). Preferably the IFE includes selectively labeling only free immunoglobulin light chain proteins, and optionally quantifying the labelled free monoclonal immunoglobulin light chain proteins. Methods of identifying a subject as having a disease or disorder associated with free monoclonal light chains, or as being at risk of having a disease or disorder associated with free monoclonal light chains including detecting and optionally quantifying the free monoclonal light chains are also described. In some embodiments, the presence of more than about 1.75 mg/L free monoclonal immunoglobulin light chain in an undiluted sample is an indication that the subject has or is at risk of a disease or disorder of monoclonal gammopathy. In some embodiments, selectively labeling free immunoglobulin light chain proteins includes contacting the protein separation profile, preferably the undiluted protein separation profile, with an antibody specific for free immunoglobulin light chain proteins, where the contacting occurs under conditions that permit binding of the free light chains with the antibody. An exemplary IFE includes the steps of (a) depositing at least one aliquot portion of the sample, preferably undiluted sample, on a deposit area of an electrophoretic gel plate having one anodic side and one cathodic side, wherein the sample deposit area is at a position of the gel plate allowing electrophoretic migration of the acidic protein content of the deposited sample, preferably undiluted sample, towards the anodic side of the gel plate, and migration of the positively charged/basic proteins towards the cathodic end of the gel plate; (b) electrophoresing the gel plate to obtain the protein separation profile, preferably undiluted protein separation profile; (c) applying at least one capture antibody to the electrophoresed gel and permitting its reaction to form precipitate and/or detectable immunocomplexes, where the capture antibody specifically binds to free immunoglobulin light chain proteins or fragments thereof; (d) removing unbound capture antibody and excess proteins; and (e) optionally but preferably staining, visualizing, scanning and/or quantitating the immunocomplexes formed in step (c). The visual pattern of stained free polyclonal light chains is distinguishable from free monoclonal light chains.

In some embodiments, the capture antibody is a polyclonal antibody. In some embodiments, the sample is a selected from an undiluted serum sample or a concentrated urine sample, or other body fluid or extract of cells or tissues. In some embodiments, the undiluted sample is a concentrated undiluted sample. In other embodiments, the undiluted sample is a non-concentrated undiluted sample. In some embodiments the sample is a lysate of bone marrow or other cells, or tissues or tumors.

In some embodiments, removal of unbound capture antibody after formation of the precipitated and/or detectable immunocomplexes in (d) includes blotting the gel to remove unbound capture antibody and incubating the gel in a wash solution. For example, in some embodiments, the blotting includes contacting the gel with blotting filter paper, optionally where the incubation includes overlaying the gel with blotting filter paper, saturating the paper with a wash solution, incubating the gel with the filter paper and the wash solution. In some embodiments, the wash solution includes saline and the incubation time is from about one minute to about five minutes, inclusive, preferably three minutes. Typically, the washing is repeated two or more times.

In some embodiments, at least one aliquot portion of the sample, preferably undiluted sample, is deposited on the gel plate as a reference which is not submitted to step (c) but is instead contacted with a fixative solution rather than with capture antibody(ies), and steps (a), (b), (d) and optionally (e) remain the same. In exemplary embodiments, six aliquot portions of the sample, preferably undiluted, sample are deposited on the gel plate in step (a), including a reference aliquot portion and three aliquot portions that are respectively contacted in step (c) with capture antibodies specific to Immunoglobulin G (IgG), Immunoglobulin A (lgA), and Immunoglobulin M (IgM), kappa light chains and lambda light chains, respectively.

In some embodiments, the detection of free monoclonal immunoglobulin light chain proteins is compared to one or more control samples, where a protein separation profile of the control samples is produced by electrophoretic migration of the protein content of the control samples, and where the control samples include one or more of a negative control, including no free monoclonal immunoglobulin light chain, and/or a positive control including a known concentration of one or more free immunoglobulin light chain, or fragments thereof.

In some embodiments, one capture antibody is a polyclonal antibody that specifically binds to free human immunoglobulin kappa light chain. In other embodiments, one capture antibody is a polyclonal antibody that specifically binds to free human immunoglobulin lambda light chain. Preferably, the capture antibody is a polyclonal antibody specific for free human immunoglobulin kappa light chain, or for free human immunoglobulin lambda light chain. The light chains typically include or are exclusively monoclonal light chains.

In some embodiments, the methods further include one or more steps of (f) analyzing and/or interpreting the IFE results and/or concluding about the health status of the subject; and optionally (g) treating the subject for a disease, for example, when the clinical, laboratory and radiologic parameters meet diagnostic criteria for multiple myeloma. In some embodiments, the methods treat a subject when the sample includes at least about 1.15 mg/L free monoclonal immunoglobulin kappa light chains and/or at least about 1.75 mg/L free monoclonal immunoglobulin lambda light chain. In some embodiments, the treatment includes chemotherapy, immunotherapy, corticosteroids, targeted therapy, radiation therapy, proteasome inhibition, monoclonal antibodies against CD38 and/or SLAM7, antibody-drug conjugate therapy, nuclear export inhibition, bisphosphonate treatment for bone disease, CAR T cell therapy, autologous stem cell transplantation (ASCT), or a combination thereof.

In some embodiments, the methods detect or identify a disease or disorder associated with monoclonal immunoglobulins, including free monoclonal light chains selected from monoclonal gammopathy of undetermined significance (MGUS), asymptomatic or smoldering multiple myeloma (SMM), multiple/plasma cell myeloma (MM), HIV/AIDS, Chronic lymphocytic leukemia, Non-Hodgkin Lymphoma, particularly Splenic marginal zone lymphoma and Lymphoplasmacytic lymphoma, Hepatitis C, Connective tissue disease such as lupus, Immunosuppression following organ transplantation, Waldenstrom macroglobulinemia, Guillain-Barre syndrome, polyneuropathy, amyloidosis or Tempi syndrome. In some embodiments, the disease or disorder associated with free monoclonal light chains is light-chain-predominant multiple/plasma cell myeloma (LCPMM) or a light chain myeloma (LCMM). In particular embodiments, the subject has previously been treated for a disease or disorder associated with monoclonal immunoglobulins or monoclonal light chains selected from multiple/plasma cell myeloma (MM), HIV/AIDS, Chronic lymphocytic leukemia, Non-Hodgkin Lymphoma, particularly Splenic marginal zone lymphoma and Lymphoplasmacytic lymphoma, Hepatitis C, Connective tissue disease such as lupus, Immunosuppression following organ transplantation, Waldenstrom macroglobulinemia, Guillain-Barre syndrome, polyneuropathy, amyloidosis or Tempi syndrome. In some embodiments, the subject has received, or is receiving treatment for a Neoplastic monoclonal gammopathy (NMG). In other embodiments, the subject has not received, or is not receiving treatment for a Neoplastic monoclonal gammopathy (NMG), e.g., MGUS and SMM, but is being monitored for progress of disease to multiple myeloma.

Typically, the detection of monoclonal immunoglobulins or monoclonal kappa light chains or monoclonal lambda chains indicates that the subject has monoclonal gammopathy or, if the patient has been treated for MM, has residual or minimal residual disease (MRD). In some embodiments, the methods detect free monoclonal kappa light chains in serum at a concentration of about 1.78 mg/L, or more than about 1.78 mg/L. In some embodiments, the methods detect free monoclonal kappa light chains at a concentration of about 1.15 mg/L or more than about 1.15 mg/L. In certain embodiments, the subject has received, or is receiving treatment using one or more monoclonal antibody therapeutics, and/or the subject has previously been screened for the presence of free monoclonal light chains in a biological sample by another technique, and wherein the result was previously found to be negative.

Methods of identifying the presence of serum free monoclonal light chains in an undiluted serum sample from a subject by immunofixation electrophoresis (FLC-Modified SIFE), can include one or more steps of (i) depositing at least one aliquot portion of the undiluted serum sample on a deposit area of an electrophoretic gel plate having one anodic side and one cathodic side, where the sample deposit area is at a position of the gel plate allowing electrophoretic migration of the protein content of the deposited undiluted serum sample towards the anodic side of the gel plate for acidic proteins and to the cathodic side for neutral and positively charged/basic proteins; (ii) electrophoresing the gel plate to obtain the undiluted serum protein separation profile; (iii) contacting the electrophoresed gel with a solution including at least one capture antibody having specificity for free immunoglobulin light chain proteins or fragments thereof, where the contacting is under conditions that permit the formation of precipitate and/or detectable immunocomplexes between the capture antibody and free immunoglobulin light chain proteins or fragments thereof within the protein separation profile; (iv) removing unbound capture antibody by blotting the solution including at least one capture antibody by contacting the gel with blotting paper; (v) contacting the gel with a wash solution including saline, and incubating the gel with the wash solution for at least one minute, preferably three minutes, then removing the wash solution; (vi) repeating step (v) from one to ten times, inclusive; and (vii) optionally, staining and/or quantitating the immunocomplexes formed in step (iii). Visual examination of the washed, stained gel allows for distinction of monoclonal light chains from polyclonal light chains.

Methods of identifying the presence of free monoclonal light chains in a urine sample from a subject by immunofixation electrophoresis (IFE) are also provided. Typically, the methods include the steps of (i) concentrating the proteins in urine. In an exemplary embodiment, the proteins are concentrated by membrane filtration to a 5 to 200 fold reduction in volume by removal of water. In some embodiments, the methods concentrate the proteins within a urine sample to a total protein concentration of at least 4 mg/dL; (ii) depositing at least one aliquot portion of the concentrated urine sample on a deposit area of an electrophoretic gel plate having one anodic side and one cathodic side, where the sample deposit area is at a position of the gel plate allowing electrophoretic migration of the protein content of the deposited concentrated urine sample towards the anodic side of the gel plate for acidic proteins and to the cathodic side for neutral and positively charged proteins; (iii) electrophoresing the gel plate to obtain the protein separation profile of the concentrated urine sample; (iv) contacting the electrophoresed gel with a solution including at least one capture antibody having specificity for free immunoglobulin light chain proteins or fragments thereof, wherein the contacting is under conditions that permit the formation of precipitate and/or detectable immunocomplexes between the capture antibody and free immunoglobulin light chain proteins or fragments thereof within the protein separation profile; (v) removing unbound capture antibody by blotting the solution including at least one capture antibody by contacting the gel with blotting paper; (vi) contacting the gel with a wash solution including saline, and incubating the gel with the wash solution for at least one minute, preferably three minutes, then removing the wash solution; (vii) repeating step (v) from one to ten times, inclusive; and (viii) optionally, staining and/or quantitating the immunocomplexes formed in step (iv). In some embodiments, contacting the gel with a wash solution includes the steps of: (I) contacting the gel with a saline wash solution; (II) incubating the gel in the wash solution for 3 min; (III) contacting the gel with blotting paper to remove the wash solution; (IV) repeating steps (I-III) twice or more times; (V) contacting the gel with blotting paper filter by overlaying the gel with the paper and saturating the paper with saline wash solution; (VI) incubating the gel in the wash solution for 3 min; (VII) removing the filter paper and contacting the gel with more blotting paper filters to remove the wash solution; and (VIII) repeating steps (V-VI) twice or more times. In some embodiments, the staining and/or quantitating the immunocomplexes in step (viii) includes drying the gel and staining the gel with a dye suitable for quantitation. Visual examination of the washed, stained gel allows for distinction of monoclonal light chains from polyclonal light chains.

Kits suitable for carrying out a method of detecting serum free light chains in a biological sample are also provided. The kits typically include one or more of (i) capture antibody(ies) specific for free immunoglobulin light chain; (ii) electrophoretic gels; (iii) negative control samples including no free monoclonal immunoglobulin light chain; (iv) blotting paper; (v) wash solution; (vi) fixative solution; (v) gel stain; (vii) positive control samples including a known amount and type of free immunoglobulin light chain; (viii) apparatus for obtaining a biological sample from a subject; and (ix) apparatus for carrying out electrophoresis, staining of gels and densitometric scanning of resulting bands for estimation of protein concentration.

### BRIEF DESCRIPTION OF THE DRAWINGS

FIGS. 1A-1J are images showing representative gels from free light chain (FLC)-modified serum immunofixation protein electrophoresis (FLC-Modified SIFE) with antisera to free kappa and lambda light chains. FIGS. 1A-1B each show one of two gel “lanes”, including a single gel band at the position of a typical monoclonal kappa light chain (FIG. 1A), and including bands at positions representing a combination of polyclonal kappa light chains plus a monoclonal light chain (FIG. 1B), respectively. FIGS. 1C-1F each show images of gels loaded with serial dilutions of serum at ratios of 1:4 (FIG. 1C), 1:8 (FIG. 1D), 1:16 (FIG. 1E), and 1:32 (FIG. 1F), respectively, each showing a band corresponding to a monoclonal lambda light chain, detectable with decreasing intensity. FIGS. 1G-1J each show a gel loaded with serial dilutions of serum at rations of 1:16 (FIG. 1G), 1:32 (FIG. 1H), 1:64 (FIG. 1I) and 1:128 (FIG. 1J), respectively, each showing a band corresponding to a monoclonal kappa light chain, detectable with decreasing intensity.

FIGS. 2A-2B show representative gels from FLC-Modified SIFE, each showing gel lanes including conventional serum immunofixation (SIFE) stained with antisera for serum protein (SP), IgG (G); IgA (A), IgM (M), Kappa light chain (κ), or Lambda light chain (k), respectively. The lanes marked anti-free k (FIG. 2A) and anti-free λ (FIG. 2B) represent FLC-Modified SIFE, stained with antisera to respective free light chains.

FIG. 3 shows a representative gel from classical SIFE stained with anti-kappa antibody, with a low intensity monoclonal kappa light chain indicated in lane A. No kappa light chain was detected with antiserum to free kappa light chains (presented in lane B).

FIGS. 4A-4C shows representative gels from conventional UIFE and FLC-UIFE from each of three patients, including Patient 1 (FIG. 4A), Patient 2 (FIG. 4B), and Patient 3 (FIG. 4C). The lanes marked SP, G, A, M, K, and L represent conventional UIFE. The unmarked separate lane in first two patients were stained with anti-serum to free kappa light chains and in the third patient for free lambda light chains. The lack of detection of free monoclonal kappa light chain in patient 1 (FIG. 4A) by conventional UIFE is due to overlap in the location of intact monoclonal IgG K and K free monoclonal light chain band. The detection of monoclonal kappa and lambda light chain bands in patients 2 (FIG. 4B) and 3 (FIG. 4C) reflects the greater sensitivity of FLC-UIFE over conventional UIFE.

FIG. 5 shows a representative gel from Conventional serum immunofixation electrophoresis (SIFE) and free light chain (FLC)—SIFE from the patient with the highest level of lambda FLCs at institution A. Lane A represented staining for IgA; lane L represents staining for lambda LCs by conventional reagents in the Helena IFE kit (Helena Laboratories); lane FL represents staining with Sebia antiserum to lambda FLCs.

### DETAILED DESCRIPTION OF THE INVENTION

### I. Definitions

The term “sample” from a subject means a tissue (e.g., tissue biopsy), organ, cells (including a cell maintained in culture), cell lysate (or lysate fraction), or body fluid from a subject. Non-limiting examples of body fluids include blood, urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid. In preferred embodiments, the biological sample of the disclosed methods is urine, or serum obtained from the subject, and lysates of bone marrow or peripheral blood cells.

The term “subject” means any individual who is the target of diagnosis or treatment administration. The subject can be a vertebrate, for example, a mammal. Thus, the subject can be a human. The term does not denote a particular age or sex. A patient refers to a subject afflicted with a disease or disorder. The term “patient” includes human and veterinary subjects.

The term “therapeutically effective” means that the amount of the composition used is of sufficient quantity to ameliorate one or more causes or symptoms of a disease or disorder. Such amelioration only requires a reduction or alteration, not necessarily elimination.

By “treat” or “treatment” is meant the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied.

The use of any and all examples, or exemplary language (e.g., “such as”) provided herein, is intended merely to better illuminate the description and does not pose a limitation on the scope of the description unless otherwise claimed. Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed. All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

### II. Improved Immunoassays for Detection of Free Monoclonal Light Chains

Methods of assaying a sample for the presence of free monoclonal Immunoglobulin Light chains with enhanced resolution can be used to identify the presence of neoplastic monoclonal gammopathies (NMG), residual or minimal residual disease associated with NMG in a subject. Therefore, methods for measuring protein levels of free monoclonal immunoglobulin kappa and/or lambda light chains in a biological sample, preferably an undiluted biological sample, from a subject are provided. In some embodiments, the method is an immunoassay. Immunoassays, in their most simple and direct sense, are binding assays involving binding between antibodies and antigen. Thus, in some aspects, the method involves detecting free monoclonal immunoglobulin kappa light chain (IgK), free immunoglobulin lambda light chains (IgL) or a combination thereof, using one or more antibodies that specifically binds IgK, or IgL or a combination thereof. The method can detect human free polyclonal and monoclonal immunoglobulin kappa light chain (IgK), free polyclonal and monoclonal immunoglobulin lambda light chains (IgL) or a combination thereof with greater resolution than other assays.

The methods are typically implemented using urine immunofixation electrophoresis, (UIFE), and/or serum protein electrophoresis (SPEP) and immunofixation protein electrophoresis (SIFE), however in some embodiments the methods are implemented using one or more of the many other types and formats of immunoassays suitable for detecting the disclosed biomarkers, including enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP), together with SIFE, or as a stand-alone assay.

In general, the immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. Typically, the sample-antibody composition within the immunoassay, such as SIFE, can then be washed to remove any unbound or non-specifically bound antibody species or other proteins, allowing only those antibodies and proteins specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label.

Immunoassays that involve the detection of a substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

A preferred immunoassay is a serum protein immunofixation electrophoresis (SIFE) assay, modified according to the described methods.

A. Protein Immunofixation Electrophoresis (IFE)

Methods of enhanced protein immunofixation electrophoresis (IFE) for the detection of free monoclonal immunoglobulin light chains in a biological sample are provided. Typically, the methods identify, quantify or monitor conditions that result in abnormal protein production or loss of protein in a subject. In exemplary embodiments, the methods identify free monoclonal immunoglobulin light chains in an undiluted biological samples, such as serum or urine, or cell and tissue lysates. In further embodiments, the methods selectively label and detect only free immunoglobulin light chains in the sample, and/or include multiple additional wash steps prior to detection and quantitation, to provide enhanced resolution of detection of free monoclonal light chains, relative to classical/conventional IFE.

The terms serum protein immunofixation electrophoresis (SIFE), Serum Protein Electrophoresis, Protein ELP, SPE, SPEP, Gel Electrophoresis, Capillary Electrophoresis, Immunosubtraction Electrophoresis, Urine Protein immunofixation Electrophoresis (UIFE), UPE, UPEP, IFE, CSF, Protein Electrophoresis, and Electrophoresis are used herein to refer to methods for separating and optionally identifying proteins on the basis of electrical charge.

Serum protein electrophoresis (SPEP) and serum Immunofixation electrophoresis (SIFE) are methods broadly used in clinical laboratories for the detection, identification, and follow-up of the progression of immunoglobulins involved in monoclonal gammopathies.

Typically, the methods include modified immunofixation electrophoresis (FLC-Modified IFE). Immunofixation electrophoresis (IFE) i.e., “classical” IFE, is a well-established method for detecting and typing certain proteins, especially monoclonal immunoglobulins or immunoglobulins in biological samples. Assayed biological samples are usually serum, urine, or cerebrospinal fluid. IFE is a two-stage procedure combining protein electrophoresis (SPEP) as a first step and immunofixation as a second step. The technique is widely used as routine analysis carried out in clinical analysis laboratories, for analyzing biological samples with a view to typing the immunoglobulins they contain. IFE provides for the identification of anomalies in different biological samples, in biological liquids such as, e.g., serum, urine or cerebrospinal fluid.

Classical IFE remains the prevalent method for immunoglobulins typing and follow-up of patients presenting with multiple myeloma, although subject to other problems. Although the interpretation of IFE results can be seen as a very qualitative exercise, subject to the experience and skills of the practitioner, the interpretation of the results of conventional IFE experiments is considered easier than that of other techniques (e.g., SPE or CE) for those skilled in the art, except in certain situations. In some examples, “conventional” or “classical” IFE does not identify free monoclonal immunoglobulin light chains in an undiluted biological sample, such as serum or urine, and/or does not selectively label and detect free monoclonal immunoglobulin light chains in the sample, and/or does not include multiple additional wash steps prior to detection and quantitation. Typically, “conventional” or “classical” IFE labels and detects multiple species of immunoglobulins in a diluted sample, does not include one or more additional wash steps to remove unbound capture antibodies, and does not provide the enhanced resolution of detection of free monoclonal immunoglobulin light chains in a sample that can be achieved according to the disclosed methods for FLC-modified IFE.

1. FLC-Modified IFE

The disclosed methods employ a modified IFE technique that provides greatly enhanced sensitivity of detection of free monoclonal immunoglobulin lights chains in a biological sample.

In some forms, the methods include FLC-Modified Serum protein Immunofixation electrophoresis (FLC-Modified SIFE). In other forms, the methods include FLC-Modified Urine protein Immunofixation electrophoresis (FLC-Modified UIFE).

It has been established that immunoglobulin monoclonal light chains (MLCs) in serum and urine are markers for monoclonal gammopathy and serve as markers of minimal residual disease (MRD) in multiple myeloma (MM). Excretion of MLCs in urine is known to result in renal damage and shorter survival in patients with LC-predominant MM.

As set forth in the Examples, urine immunofixation results and medical records validated the concept that detection of monoclonal free light chains using FLC modified IFE is significantly more sensitive and more efficient than conventional methods for detecting MLCs in urine or serum.

Typically, the methods for detecting free monoclonal immunoglobulin light chains in a biological sample by FLC-Modified IFE include the steps of:
(1) Providing an undiluted serum sample, or a concentrated urine sample or cell lysate;
(2) Protein electrophoresis of the undiluted sample;
(3) Immunofixation with capture antibodies specific for free kappa light chains, (IgK), or free lambda light chains (IgL), or both; and
(4) Protein identification/quantitation.

In some embodiments, one or more composition or steps for FLC-Modified SIFE is based on or includes one or more of the compositions or steps described in Wilhite, et al., Practical Laboratory Medicine, 27, (2021), e00256, the content of which is specifically incorporated herein in its entirety.

i. Providing an Undiluted Sample

It will be appreciated that the disclosed methods typically include assaying a biological sample. Although assaying diluted samples accordingly to the disclosed methods are contemplated and thus expressly disclosed for all the assays herein (e.g., in place of an undiluted sample in any of the methods as disclosed herein), preferred embodiments feature an undiluted sample. Undiluted samples are minimally processed, for example, to remove any insoluble/solid component from the sample, without altering the physiological concentration of soluble proteins. Undiluted samples can be concentrated or unconcentrated.

Thus, in preferred embodiments, the methods include providing an undiluted sample, such as an undiluted serum sample or concentrated urine sample for electrophoresis. Typically, the undiluted serum sample is a biological sample that has been minimally processed, for example, to remove any insoluble/solid component from the sample, without altering the physiological concentration of soluble proteins. Typically, the methods obtain a biological sample, such as a serum or urine sample from a subject and optionally include one or more steps to process the biological sample to remove the insoluble components, such as cells and debris, whilst retaining 100% or nearly 100%, such as 99%, 98%, 97%, 96%, 95%, 94%, 93%, 02%, 91% or 90% of the physiological concentration of the soluble proteins, such as immunoglobulins, immunoglobulin light chains or fragments thereof, within the sample. In some embodiments, a sample loaded onto a lane of an electrophoresis gel is a portion that is less than 100% of the total amount of a biological samples, such as a serum or urine sample, obtained from a subject. Therefore, in some embodiments, the methods provide one or more samples that are an aliquot from an undiluted serum or concentrated urine sample from a subject. In some embodiments, the methods include one or more positive or negative controls in addition to the experimental samples. This is in contrast to conventional SIFE in which serum sample is diluted 5 to 10-fold before application for electrophoresis.

In some embodiments, the methods include one or more steps of concentrating the biological sample and extraction of soluble proteins from tissue or cells, such as bone marrow cells. In an exemplary embodiment, the methods concentrate a biological sample (i.e., concentrated biological sample), such as a sample of serum or urine from a subject. The concentration can include any method known in the art to concentrate a sample, such as a biological sample. Preferably, the methods of concentration do not remove free light chains from the sample. For example, in some embodiments, the methods remove a portion of the solution from the sample without or minimally removing or altering the protein components. In some embodiments, the methods remove a portion of the solution from the sample without or minimally removing or altering the protein components of more than a size limit, and/or having a particular charge or hydrodynamic volume. Typically, the concentrated sample contains a greater concentration of one or more protein components than is the physiological or “natural” concentration of the protein components. In an exemplary embodiment, the methods concentrate the sample using filtration, for example, to remove a portion of the liquid from the sample, while retaining the proteins within the sample. In an exemplary embodiment, the methods use a filtration device configured to retain proteins having a specific molecular weight, i.e., corresponding to the weight of a free immunoglobulin light chain protein. The methods for concentration can be determined according to the nature of the sample that is to be concentrated. For example, in some embodiments, the sample is or contains urine, and the sample is concentrated by membrane filtration, for example, using Millipore urine concentrators. In some embodiments, the proteins in a sample are concentrated by chemical precipitation and precipitate re-dissolved in saline to constitute a specimen for further analysis.

When the methods include one or more steps to concentrate the sample, the sample is concentrated to provide a concentrated sample having a protein concentration that is equal to or greater than the initial protein concentration of the sample prior to concentration. For example, in some embodiments the methods increase the concentration of the free light chain proteins in the sample by a specific amount, such as from about 0.1% to about 100,000%, inclusive, of the initial concentration of the free light chain proteins in the same sample prior to the concentration. In some embodiments the concentrated sample includes about 101% to about 100,000%, inclusive, of the concentration of free light chain proteins in the original sample prior to the concentration.

ii. Protein Electrophoresis

The methods typically include one or more steps of protein electrophoresis to obtain a protein separation profile of the protein sample. In the first step of IFE, electrophoresis of the protein content of an undiluted biological sample is performed on an electrophoretic support (usually a gel) under an applied electric field. This allows protein fraction(s) separation (resolution) in the form of an electrophoretic profile, such as a “separation profile” for the proteins within a sample.

Gel protein electrophoresis exploits the fact that proteins have an intrinsic electrical charge. When applying an electric field, the intrinsic charge of a given protein imparts an electrophoretic mobility to the protein and thus permits its migration in the gel toward an electrode having a charge opposite to the charge of the protein. As an undiluted biological sample contains several protein types, proteins having lower electrophoretic mobility will move slower than those with higher electrophoretic mobility and hence separation of the proteins of the biological sample from one another can be achieved. All types of conventional electrophoretic gel types can be used for the described methods. In a particular embodiment, the electrophoretic gel plate corresponds to a high-resolution gel, such as an agarose gel, which shall improve the resolution in the beta and gamma zones of the gel. Suitable agarose gels are known in the art. Exemplary agarose gels have a concentration of agarose from 0.5% to 2%. In a particular embodiment, the concentration of agarose is 0.8%. Other type of gels can however be used, including acrylamide gels.

In an exemplary embodiment, the methods include:
(a) depositing at least one aliquot portion of an undiluted biological sample on a deposit area, such as one or more “lanes” of an electrophoretic gel plate having an anodic and cathodic side, wherein the sample deposit area is preferably at a position of the gel plate allowing electrophoretic migration of the acidic protein content of the deposited sample towards the anodic side of the gel plate; and positively charged proteins to the cathodic side, and
(b) electrophoresing the gel plate to obtain an electrophoresed gel plate including a protein separation profile of the undiluted biological sample deposited in step (a), wherein the separation profile includes displacing specifically an immunoglobulin and/or fragment thereof that may be present in the biological sample during the electrophoretic migration.

The quantity of electric charges of a protein or fragment thereof, such as a free light chain, can be estimated through the determination of the electrophoretic mobility of said protein or free light chain. The electrophoretic mobility of a molecule (μep) is directly proportional to the net electric charge of the molecule, according to the Debeye-Huckel-Henry equation μep=q/6(pi)ηR, wherein q is the net electric charge of the molecule, η is the viscosity of the medium and R is the ionic radius of the molecule.

In an exemplary embodiment, electrophoresis is carried out in buffer solution(s) commonly used in the art and for IFE, such as barbital, or TrisNeronal buffer, at conventional pH(s), for example using a barbital buffer at pH 8.6, during a conventional time for carrying IFE according to usual protocols, such as 15 minutes or less, and at a conventional temperature, such as or at a cooler temperature such as 4° C. These parameters can readily be adjusted according to the practice or recommendations of the manufacturer(s) of IFE devices. In a particular embodiment, the migration of samples and modified antibody is carried out in 15 minutes or less at 20 Watts. According to a particular embodiment, the migration of samples and modified antibody is carried out at 4° c. According to a particular embodiment, the migration of samples and modified antibody is carried out in 15 minutes or less at 20 Watts and at 4° C.

Typically, the electrophoresis is carried out under conditions that do not alter the structure of the proteins within the sample. For example, the electrophoresis is carried out under conditions that preserve the native state of the immunoglobulins and free light chains within the sample.

iii. Immunofixation

The methods typically include one or more steps of immunofixation to selectively label the free immunoglobulin light chains within the protein separation profile.

In the second step of IFE, immunofixation is performed to permit the detection and typing of the monoclonal immunoglobulins, or fragments thereof that may be present in the assayed sample. Typically, several aliquots of the same undiluted biological sample are deposited in parallel on agarose gel. After the electrophoresis of the first step, each electrophoresed track is incubated with a type of antibody that is specific to the types of immunoglobulin or immunoglobulin light chains being investigated. (In the FLC-Modified SIFE, antibodies to free immunoglobulin kappa light chain (IgK), and/or free immunoglobulin lambda light chain (IgL) are also employed), leading to the formation of immunocomplexes between the monoclonal immunoglobulin or monoclonal light chains in the sample and the antibodies. It may be that non-monoclonal or polyclonal immunoglobulins and immunoglobulins light chains also react with the reagent antibody/antiserum, however the pattern of staining of polyclonal and monoclonal immunoglobulins, polyclonal light chains and monoclonal light chains are distinguishable. In preferred embodiments, the methods contact the proteins within the protein separation profile with one or more capture antibodies that selectively bind to polyclonal and monoclonal immunoglobulins or free monoclonal and polyclonal immunoglobulin kappa light chain and/or free monoclonal and polyclonal immunoglobulin lambda light chain, under conditions that allow formation of immunocomplexes between the immunoglobulins and immunoglobulin light chains in the sample and the capture antibodies.

Typically, a fixative solution (for electrophoresed reference track) and antisera including capture antibodies which are specific for different immunoglobulin classes and types (e.g., IgG, IgA, Ig M, IgK, IgL [and in the case of FLC-Modified SIFE, antibodies to free IgK and free IgL]) are applied to determined tracks of the gel. The gel, fixative solution and these different antisera (capture antibodies) are incubated during a time during which immune complexes are formed between the specific immunoglobulins including IgK, and/or IgL and the capture antibodies.

Therefore, in some embodiments, the methods include:
(c) applying at least one capture antibody on an appropriate region of the electrophoresed gel plate, wherein the capture antibody has specificity for a particular (determined) immunoglobulin isotype, immunoglobulin heavy chain or immunoglobulin light chain, or has specificity for a target immunoglobulin or fragment thereof, or has specificity for a particular (determined) immunoglobulin isotype and/or target immunoglobulin or fragment thereof as found in an immunocomplex between the target immunoglobulin or fragment thereof and the capture antibody, and permitting its reaction to allow the formation of precipitate and/or detectable immunocomplexes.

The FLC-Modified SIFE uses undiluted serum sample and applies antisera specific to free light chains. The antibodies to light chains in conventional/classical IFE react with both free light chains and light chains bound to heavy chains. Conventional SIFE typically uses 5 to 10-fold diluted serum samples. The antisera used in FLC-Modified SIFE reacts with only free light chains.

The light chains typically include both free monoclonal light chains and free polyclonal light chains. Thus, in some embodiments, free monoclonal and free polyclonal light chains are captured. In other embodiments, only free monoclonal light chains are captured.

In some embodiments the capture antibodies are specific for free immunoglobulin kappa light chain (IgK), and/or free immunoglobulin lambda light chain (IgL). Typically, the staining patterns allow distinction between the reactivities of polyclonal and monoclonal immunoglobulins and immunoglobulin light chains.

Capture antibody(ies) can be specific for one or more target immunoglobulin or a fragment thereof, or the determined antibody isotype. In a particular embodiment, capture antibody(ies) is(are) specific for the target immunoglobulin or fragment thereof, especially a human target immunoglobulin or fragment thereof.

Typically, the immunofixation procedure following the electrophoresis uses common anti-immunoglobulins sera (capture antibody(ies)) for typing purposes. Anti-immunoglobulins sera (capture antibody(ies)) can be of human or non-human animal origin. In preferred embodiments, the capture antibody(ies) are of non-human origin, such as rabbit, sheep or mouse animal origin. In a particular embodiment, capture antibody(ies) are rabbit antibody(ies). The capture antibody(ies)) can recognize a particular antibody isotype in order to reveal its presence. The capture antibody(ies)) can also recognize the target immunoglobulin or fragment thereof as defined herein (especially monoclonal immunoglobulin). In a particular embodiment, capture antibody(ies)) recognize a soluble target immunoglobulin or fragment thereof that does not precipitate or is not found in a precipitated form in an electrophoretic gel, especially an electrophoretic gel used for carrying out the described methods. In a particular aspect, incubation time on the gel of capture antibody(ies) is about 5 minutes, for example, 1, 2, 3, 4, 5, 6, 7, 8, 9 or 10 minutes. Incubation times can be readily adapted by the skilled person seeking a better sensitivity, according to standard practice in the field.

iv. Wash Steps

The methods include one or more wash steps to remove unbound capture agents and/or proteins and from the gel. In conventional IFE, typically, the wash steps include one round of (i) contacting the electrophoresed gel with (a) filter paper shaped to fit the antibody slots and (b) one set of two filter papers. In some embodiments the method includes one or more steps of (i) contacting the electrophoresed gel with a first suitable protein absorbing matrix, (ii) applying a suitable wash buffer, (iii) incubating the gel in the wash solution for a suitable period of time, (iv) removing the wash buffer, and (v) contacting the electrophoresed gel with a second or further suitable protein absorbing matrix.

An exemplary first or second or further protein absorbing matrix for use in (i) and/or (v) is a filter paper. In some embodiments, the matrix is a filter paper that is shaped to fit the antibody slots. In some embodiments, the methods include contacting the electrophoresed gel with multiple filter papers. The filter papers can be the same or different types and sizes. Therefore, in some embodiments, the methods include contacting the gel with two, three, four, five or more different types of filter paper. Typically, the one or more rounds of contacting the gel with filter paper removes all or most of the unbound capture antibodies and proteins for the gel.

Typically, the gel contacted with the filter paper is contacted in a suitable wash solution in (ii). An exemplary wash solution is saline. Suitable volumes of wash solution include from about 10 μL to about 500 μL, inclusive, such as 50-100 μL, inclusive.

Typically the gel is incubated with the wash solution in (iii) for a suitable period of time to allow transfer of the proteins to the filter paper, such as one, two, three or more minutes. The methods optionally include one or more steps of removing the excess wash solution using a suitable matrix, such as a blotting paper. In some embodiments, the methods apply blotting paper after each application of the wash solution. The wash solution, incubation and optional removal step can be applied once, twice, three times, four times, five times, or more than five times. In a preferred embodiment, the methods apply three rounds of wash buffer, incubation and blotting.

In some embodiments, the methods apply a second protein absorbing matrix, such as a second piece of filter paper in (v), following the one or more wash and blotting steps. The second or further filter papers can be the same or different types and sizes. Therefore, in some embodiments, the methods include contacting the gel with two, three, four, five or more different types of filter paper. In some embodiments, the methods repeat the washing steps (i) through (v) once or more than once, such as twice, or three times, or more than three times.

In an exemplary embodiment, the wash steps include removing unbound capture antibody and proteins by soaking the excess antibody with filters in the kit, shaped to fit the antibody slots; three steps of contacting the gel with 50 μL of saline, incubating for three minutes, removing excess saline with blotting paper, repeating the process two time and with two sets of filter papers, separated by contacting the gel with a thin filter paper soaked in saline and repeating the process two times.

In some embodiments, prior to a wash, the methods contact the electrophoresed gel with a suitable blotting filter paper following the immunofixation step to remove unbound capture antibody. For example, in some embodiments, the methods contact the electrophoresed gel with a blotting paper to remove the unbound capture antibody from the gel immediately following the incubation period with the capture antibody.

In some embodiments, the methods contact the blotted electrophoresed gel with a wash solution following the blotting step to remove unbound proteins and to wash any residual unbound proteins way from the gel. For example, in some embodiments, the methods incubate the electrophoresed and blotted gel with a wash solution for a period of time that is approximately 1 minute, or 2, 3, 4, 5, 6, 7, 8, 9, 10, 11, 12, 15 or 20 minutes, or more than 20 minutes. Following incubation in the wash solution, the wash solution is removed, for example, by exposure to suitable blotting filter papers. In some embodiments, the methods repeat the wash and/or blotting steps one or more times. For example, in some embodiments, the methods repeat the wash step 1 time, or 2, 3, 4, 5, 6, 7, 8, 9, 11, 12, 15 or 20 times, or more than 20 times. In preferred embodiments, the wash solution includes saline solution. In preferred embodiments, the incubation time is about three minutes.

In some embodiments, washing and blotting includes steps of contacting the gel with filter blotting paper by overlaying the gel with the filter paper, saturating the filter paper with a wash solution, such as a saline wash solution, incubating for three minutes, removing the blotting filter paper, then blotting the gel with more filter paper, and repeating the blotting once or more times, such as 2, 3, 4, 5 6, 7, 8, or 9 or 10 or more than ten times.

An exemplary method includes the following set of wash steps:
(i) Following incubation with antiserum the antiserum is blotted with SIFE filters (such as those in Helena SIFE kits);
(ii) following blotting of excess antisera, 50 μL of saline wash solution is added to the electrophoresed gel and incubated for 3 min;
(iii) following washing, wash solution is blotted away from the gel;
(iv) washing and blotting steps (ii-iii) are repeated twice;
(v) the blotted and washed gel is subjected to blotting with two more filter papers (such as filter “C” and filter “D”, according to the Helena manufacturer's protocol); and
(vi) the gel is overlaid with fresh blotting filter paper (such as filter “C” according to the Helena manufacturer's protocol) and the blotting filter paper is flooded with saline and incubated for 3 min.
(vii) the blotting filter paper is removed followed by blotting with filters (such as filter “C” and “D” according to the Helena manufacturer's protocol).
(viii) the process of contacting the gel with filter paper, overlaying with saline and incubating for 3 minutes, followed by blotting with additional filter papers and blotting in (vi)-(v) is repeated twice.

v. Protein identification/quantitation.

After washing the gel to eliminate non-precipitated proteins, a staining step can reveal the position of the immunocomplexes: in the absence of monoclonal proteins, only a diffuse stained background appears (corresponding to a multitude of immunoglobulins, constituting the “polyclonal background”); in the presence of monoclonal proteins, stained bands are revealed as sharp well-defined bands in specific regions of the gel. The locations of such immune complexes on the gel are typically visualized by staining the gel. As a result, the presence of a specific band is generally indicative of the presence of a monoclonal protein corresponding to a particular immunoglobulin class and type.

Therefore, in some embodiments, the methods include an optional step of
(e) optionally, staining the immunocomplexes formed in step (c).

Typically, staining is carried out according to conventional methods, for example with one or more of amido black, and Coomassie blue reagent(s). Staining can also be achieved using a marker linked to the capture antibody. The marker may be, dye, fluorescent compound, gold or an enzyme. The target immunoglobulin or fragment thereof recognized and bound by the capture antibody may be an immunoglobulin or fragment thereof associated with a pathological condition such as pathological monoclonal components.

The use of a negative control or reference track on which no antiserum is applied, allows the typing of each monoclonal band that is visible on the gel, by comparison with the reference track. Immunoglobulins are generally formed from heavy chains (2 heavy chains) and light chains (2 light chains). Five heavy chain isotypes (M, G, A, D, E″ isotypic classes) and two light chain isotypes (kappa and lambda isotypic types) have been identified in that four-chain structure.

In preferred embodiments the methods include (i) staining of the gel; and (ii) densitometric scanning of the stained gel to identify and quantify the light chain proteins present. In an exemplary embodiment, the relative area under the monoclonal peak, compared to that of the total involved light chain composition, was estimated by densitometric scanning of immunofixation gels. The proportion of the area occupied by the monoclonal peak in representative densitometric scans was used to arrive at the total serum concentration of the monoclonal serum free light chains (G. Singh, Bollag R quantification by ultrafiltration and immunofixation electrophoresis testing for monoclonal serum free light chains, Lab. Med. 51 (2020) 592-600.)

vi. Detecting Diseases and Disorders

In some embodiments, the methods also include one or more step of analyzing and/or interpreting the IFE results and/or concluding about the health status of the patient, for example, based on results other laboratory, cytogenetic, and radiologic examinations, of the biological sample of which has been subjected to the method.

The disclosed methods include the determination, identification, indication, correlation, diagnosis, prognosis, etc. (which can be referred to collectively as “identifications”) of subjects, diseases, conditions, states, etc. based on imaging, measurements, detections, comparisons, analyses, assays, screenings, etc.

Therefore, in some embodiments, the methods include one or more steps of
(f) analyzing and/or interpreting the IFE results and/or concluding about the health status of the subject.

For example, the disclosed methods allow identification of patients, organs, tissues, etc. having a disease or disorder. Such identifications are useful for many reasons. For example, and in particular, such identifications allow specific actions to be taken based on, and relevant to, the particular identification made. For example, diagnosis of a particular disease or condition in particular subjects (and the lack of diagnosis of that disease or condition in other subjects) has the very useful effect of identifying subjects that would benefit from treatment, actions, behaviors, etc. based on the diagnosis. For example, treatment for a particular disease or condition in subjects identified is significantly different from treatment of all subjects without making such an identification (or without regard to the identification). Subjects needing or that could benefit from the treatment will receive it and subjects that do not need or would not benefit from the treatment will not receive it.

Accordingly, also disclosed are methods including taking particular actions following and based on the disclosed identifications. For example, disclosed are methods including creating a record of an identification (in physical—such as paper, electronic, or other—form, for example). Thus, for example, creating a record of an identification based on the disclosed methods differs physically and tangibly from merely performing an imaging, measurement, detection, comparison, analysis, assay, screen, etc. Such a record is particularly substantial and significant in that it allows the identification to be fixed in a tangible form that can be, for example, communicated to others (such as those who could treat, monitor, follow-up, advise, etc. the subject based on the identification); retained for later use or review; used as data to assess sets of subjects, treatment efficacy, accuracy of identifications based on different measurements, detections, comparisons, analyses, assays, screenings, etc., and the like. For example, such uses of records of identifications can be made, for example, by the same individual or entity as, by a different individual or entity than, or a combination of the same individual or entity as and a different individual or entity than, the individual or entity that made the record of the identification. The disclosed methods of creating a record can be combined with any one or more other methods disclosed herein, and in particular, with any one or more steps of the disclosed methods of identification.

As another example, disclosed are methods including making one or more further identifications based on one or more other identifications. For example, particular treatments, monitoring, follow-ups, advice, etc. can be identified based on the other identification. For example, identification of a subject as having a disease or condition with a high level of a particular component or characteristic can be further identified as a subject that could or should be treated with a therapy based on or directed to the high-level component or characteristic. A record of such further identifications can be created (as described above, for example) and can be used in any suitable way. Such further identifications can be based, for example, directly on the other identifications, a record of such other identifications, or a combination. Such further identifications can be made, for example, by the same individual or entity as, by a different individual or entity than, or a combination of the same individual or entity as and a different individual or entity than, the individual or entity that made the other identifications. The disclosed methods of making a further identification can be combined with any one or more other methods disclosed herein, and in particular, with any one or more steps of the disclosed methods of identification.

Depending on the diseases of the investigated patients, monoclonal proteins that can be identified are of a different nature, constituted either by an intact antibody/immunoglobulin molecule, or by a fragment of antibody. Thus, heavy chains or light chains can be produced alone. This is the case, for example, with Bence Jones proteins secreted in the urine of patients with myelomas, which are in the form of light chains alone. The isotypes that are to be determined for the immunoglobulins can be characterized as a function of the nature of their heavy chains and/or as a function of the nature of their light chains.

The term “monoclonal protein” refers to heavy chains of a single isotypic class (and possibly subclass) and light chains of a single isotypic type, either singly or in the usual form of a tetrameric molecule.

A biclonal gammopathy will typically present as two bands of heavy chain (identical or different) and two bands of light chains (identical or different) when seen by immunofixation. For example, a biclonal pattern may consist of IgG Kappa and IgA Kappa monoclonal immunoglobulins or IgG Kappa and IgG lambda monoclonal immunoglobulins.

If an oligoclonal gammopathy is present, multiple, possibly weak bands of two or more types of heavy chains and one or two types of light chains will typically be seen. However, detection of an oligoclonal gammopathy in the presence of a significant polyclonal background may be part of a normal response to various stimuli, including stem cell transplantation. Similarly, in the presence of a polyclonal background, especially when the analyzed sample is diluted to minimize the interference of said polyclonal background on antisera (capture antibodies) tracks, the monoclonal protein that the analyzed sample may contain may also be diluted so as to render it invisible in the polyclonal background. In this case, the possibility of the presence of monoclonal protein cannot be excluded. The use of undiluted serum and using polyclonal antisera specific for free light chains according to the methods is instrumental in ascertaining monoclonal free light chains and thus the presence of a pathological state.

The presence of monoclonal component(s) in a biological sample is characteristic of an excessive production of one single type of immunoglobulin belonging to a class selected amongst lgG, lgA, lgM, lgD or lgE, as well as kappa chain, or lambda chain, free kappa chain or free lambda chain. Monoclonal component(s) arise from the proliferation of one specific clone of malignant, terminally differentiated B cells which in turn generates a homogenous population of monoclonal immunoglobulins.

In preferred embodiments, the methods identify the presence of free monoclonal immunoglobulin kappa light chain (IgK), and/or free monoclonal immunoglobulin lambda light chain (IgL) within the undiluted serum sample.

Presence of therapeutic antibodies in a sample is normally associated with a medicinal treatment of a patient that may be unknown to the practitioner in charge of the IFE analysis.

For some lgA gammopathies, the anti-light chain antiserum may present a faint affinity with the corresponding monoclonal immunoglobulin, and its detection is more difficult. In that case, it is recommended to test the sample with a Bence Jones immunofixation procedure where the antiserum reaction is amplified due to a longer incubation time. Alternatively, mild reduction of the IgA may expose light chain epitopes by abrogating the dimeric structure of IgA.

For some lgD gammopathies, the anti-light chain antiserum may present a faint affinity with the corresponding monoclonal immunoglobulin.

For some lgE gammopathies, the anti-light chain antiserum may present a faint affinity with the corresponding monoclonal immunoglobulin.

Several illnesses can present with a monoclonal gammopathy, such as Monoclonal gammopathy of undetermined significance (MGUS), smoldering multiple myeloma, Multiple myeloma, but also AIDS, Chronic lymphocytic leukemia, Non-Hodgkin Lymphoma, particularly Splenic marginal zone lymphoma and Lymphoplasmacytic lymphoma, Hepatitis C, Connective tissue disease such as lupus, Immunosuppression following organ transplantation, Waldenstrom macroglobulinemia, Guillain-Barre syndrome, polyneuropathy, amyloidosis or Tempi syndrome.

a. Multiple Myeloma (MM)

In some embodiments, the methods identify multiple myeloma (MM) in a subject. In about 85% of MM cases, the tumors secrete intact immunoglobulins. About 15% of MM secrete only light chains. However, in almost all instances of MM secreting intact immunoglobulins, an excess of free monoclonal light chains is also secreted. While excess free monoclonal light chains may be detectable in serum or urine by conventional IFE, the described method, FLC-Modified SIFE, detects free monoclonal light chains with greater sensitivity. Excess free monoclonal light chains can also be detected in urine and detection of monoclonal light chains is the main reason for performing UIFE. In some embodiments, the methods identify light chain MM (LCMM) in a subject. The methods including SFLC quantification are useful in the diagnosis and monitoring of light chain myelomas (LCMM). In some embodiments, the methods identify light chain predominant MM (LCPMM) in a subject. About 18% of the intact immunoglobulin secreting MM tumors produce a greater abundance of free monoclonal light chains and this group has been defined as light chain predominant MM (LCPMM). This sub-group has shorter survival probably due to renal damage inflicted by excess free monoclonal light chains (G. Singh, et al., Light chain predominant intact immunoglobulin monoclonal gammopathy disorders: shorter survival in light chain predominant multiple myelomas, lmaa057, Lab. Med. (2020 Nov. 12), doi.org/10.1093/labmed/lmaa057; Singh, et al., Light chain-predominant multiple myeloma subgroup; Impaired renal function correlates with decreased survival in this subgroup. Lab Med. 2021; 53:145-148 limab054).

Patients with LCPMM and light chain MM (LCMM) patients with tumors secreting higher levels of free monoclonal light chains exhibit greater renal injury and a two-year shorter survival than conventional MM.

b. Minimal Residual Disease

In some embodiments, the methods identify Minimal residual disease (MRD) relating to a MM following treatment. In a preferred embodiment, the methods identify Minimal residual disease (MRD) of multiple myeloma (MM) in a subject. Minimal residual disease of multiple myeloma refers to the small number of malignant cells below the limit of detection available with conventional morphologic assessment. In multiple myeloma, MRD refers to myeloma cells that are present in the bone marrow after a clinical response has been measured and the patient is in complete remission. The current criterion for minimal residual disease is one or more than one neoplastic MM cell per 10⁶ nucleated cells in the bone marrow, in a patient who meets criteria for complete remission/stringent complete remission.

c. Resolution of Detection

The methods provide enhanced resolution of detection of free monoclonal immunoglobulin light chains in a biological sample, as compared with prior methods. In some embodiment, the methods identify free monoclonal immunoglobulin light chains indicative of Minimal residual disease (MRD) of multiple myeloma (MRDMM) in the same or different sample from a subject that was previously screened by a different method. In preferred embodiments, the prior screening by a different method did not identify free monoclonal immunoglobulin light chains in the same undiluted biological sample, or did not identify as much free monoclonal immunoglobulin light chains in the same undiluted biological sample. Therefore, in some embodiments, the methods provide higher resolution of detection of monoclonal immunoglobulin and/or free monoclonal light chains in an undiluted biological sample. A preferred sample is an undiluted serum or undiluted concentrated urine sample.

Multiple/plasma cell myeloma (MM) is a malignant tumor of plasma cells and is generally characterized by synthesis and secretion of monoclonal immunoglobulins by tumor cells, and is diagnosed by a combination of testing for monoclonal immunoglobulins in serum, urine, bone marrow examination by morphology and cytogenetics, flow cytometric analysis, imagining studies and other laboratory parameters. In some embodiments, the methods detect kappa monoclonal light chains, at a concentration of at least 1.78 mg/L in serum. In some embodiments, the methods detect lambda monoclonal light chains at a concentration of at least about 1.15 mg/L. Therefore, methods for detecting as little as 1.78 mg/L of kappa monoclonal light chains and/or as little as 1.15 mg/L of lambda monoclonal light chains in an undiluted sample from a subject are provided. In some embodiments, these methods and mass-spectrometry detect minimal residual disease in a subject who has undergone treatment. In some embodiments, the methods detect minimal residual disease in a subject that has previously been identified as disease-free, i.e., in complete or stringent complete remission by one or more other methods. For example, the FLC-Modified SIFE methods can detect lambda monoclonal light chains in a serum sample of a subject having a total concentration of light chains of at least 1.2 mg/L, such as at least 1.3, 1.4, 1.5, 1.6, 1.7, 1.8, 1.9, 2.0, 2.5, or at least 3.0 mg/L lambda monoclonal light chain in the serum. The methods can detect kappa monoclonal light chains in a serum sample of a subject having a total concentration of free light chains of at least 1.8 mg/L, such as at least 1.9, 2.0, 2.5, or at least 3.0 mg/L kappa monoclonal light chain in the serum.

The concentrations of total free light chains contain both monoclonal and polyclonal light chains, hence the FLC-Modified SIFE can detect monoclonal kappa and monoclonal lambda light at concentrations far lower than 1.8 mg/L and 1.2 mg/L for kappa and lambda, respectively.

The methods can detect monoclonal light chains in a urine sample for a subject having a total concentration of at least 0.48 mg/dL, such as at least 0.56 mg/L, such as 0.6, 0.75, or at least 1.0 mg/L for monoclonal kappa free light chain in the urine, and at least 1.03 mg/L, such as 1.1, 1.25, or 1.5 mg/L for monoclonal lambda free light chain in the urine. The total light chain concentration includes both polyclonal and monoclonal light chains in a given sample.

In an exemplary embodiment, the subject has already got a primary diagnosis. For example, in some embodiments, the subject has a primary diagnosis of IgG kappa MM. In other embodiments, the subject has a primary diagnosis of IgG lambda MM. In some embodiments, the subject has a primary diagnosis that was determined by one of various methods including SPEP, SIFE, UPEP, UIFE, Bone marrow examination and in one institution by Mass Spectrometric analysis following nanobody mediated concentration of immunoglobulins. In other embodiments, the subject has a primary diagnosis that was determined by conventional (“classical”) SIFE. Therefore, in some embodiments, the methods detect the presence of monoclonal immunoglobulins and/or light chains in an undiluted sample, such as an undiluted serum or urine sample, with greater resolution, sensitivity, and specificity than is possible using other methods such as MASS-FIX/MALDI, or by conventional (“classical”) SIFE, or combinations thereof. For example, in some embodiments the method identifies the presence of monoclonal immunoglobulins and/or monoclonal light chains in an undiluted serum sample, or concentrated urine sample, where the presence of the monoclonal immunoglobulins and/or monoclonal light chains in the same sample is not detected using other methods such as MASS-FIX/MALDI, or by conventional (“classical”) SIFE, or combinations thereof. In some embodiments, the methods detect the presence of monoclonal immunoglobulins and/or monoclonal light chains in an undiluted sample, such as an undiluted serum or urine sample, or concentrated urine sample, at a concentration of one hundredth, one fiftieth, one twentieth, one tenth, one ninth, one eighth, one seventh, one sixth, one fifth, one fourth, one third, or half or a quarter of the concentration that is necessary for detection by other methods, such as MASS-FIX/MALDI, or by conventional (“classical”) SIFE, or combinations thereof.

The methods can involve assaying for the levels of one or more biomarkers disclosed in a sample from the subject. In some embodiments, a change in biomarker levels in the sample compared to a control level is an indication that the subject is at risk of developing, and/or has developed MM. In other embodiments, the detected presence of biomarker levels in the sample compared to a control level is an indication that the subject has MM, or has residual disease related to MM, or other disorders such as monoclonal gammopathy of undetermined significance (MGUS), asymptomatic or smoldering multiple myeloma (SMM) minimal residual disease (MRD).

Thus, the subject can in some embodiments be any human for which a diagnosis or prognosis relating to MM is desired or warranted. In preferred embodiments of these methods, the sample is urine, blood, plasma, serum, or bone marrow isolated from the subject. In some embodiments, the subject has one or more of the symptoms of MM, minimal residual disease (MRD) and/or a disease related to and preceding MM, such as monoclonal gammopathy of undetermined significance (MGUS), asymptomatic or smoldering multiple myeloma (SMM). In some embodiments, the subject has one or more family members or relatives with Type 1 Diabetes (T1D). Further, combinations of each and every disclosed biomarker is contemplated for use in the disclosed methods.

d. Monoclonal Antibody Therapeutics

In some embodiments, the methods identify a disease or disorder relating to the presence of serum free monoclonal immunoglobulin light chain in the undiluted serum or urine, or concentrated urine of a subject who has received, or who is receiving monoclonal antibody therapy, and/or who has exogenous monoclonal IgG Kappa in the blood. While treatment with therapeutic monoclonal antibodies can lead to detection of monoclonal immunoglobulin in serum or urine, therapeutic monoclonal antibodies do not produce free monoclonal light chains in body fluids.

Monoclonal antibody therapeutics are increasingly being used in numerous medical disciplines including allergy immunology, gastroenterology, haematology, oncology, rheumatology, and dermatology and organ transplantation. In this context, drug interference on serum IFE performed on samples collected from treated patients or spiked serum samples has been described with a number of therapeutic monoclonal antibodies. On the other hand, the presence of monoclonal antibody therapeutics may also lead clinicians to falsely suspect conditions such as monoclonal gammopathy of undetermined significance (MGUS). As clinical laboratories are rarely provided with extensive patient history, it is likely that faint monoclonal components unknowingly due to monoclonal antibody therapeutics are being reported.

Monoclonal antibodies designed for therapeutic use usually belong to the lgG Kappa class. Also, therapeutic monoclonal antibodies may encompass antibodies with other structures than that of naturally occurring antibodies. They can be human, humanized murine or chimeric antibodies, or variants thereof, especially chemically engineered variants or a vector monoclonal antibody, for example coupled to a drug. Therapeutic monoclonal antibodies can be whole (full) monoclonal antibodies, Fab fragments, F(ab′)2 fragments, scFv (single-chain variable fragment), di-scFv (dimeric single-chain variable fragment), sdAb (single-domain antibody), bispecific monoclonal antibodies such as bifunctional antibody or chemically linked F(ab′)2 fragments and also BiTE (bi-specific T-cell engager). Exemplary therapeutic monoclonal antibodies include Adalimumab, Trastuzumab, Ofatumumab, Siltuximab, Rituximab, Bevacizumab, lnfliximab, Cetuximab and Efalizumab, Natalizumab, Panitumumab, Tolicizumab, Clenoliximab, Etaracizumab, Visilizumab, Elotuzumab, Nimotuzumab, Ramicirumab, Elotuzumab, Daratumumab, Mapatumumab, Golimumab, Ustekinumab, Nivolumab, and functionally equivalent antibodies, i.e., antibodies having the same antigenic target, or any mixture thereof.

vii. Treating a Subject

In some embodiments, the methods include the step of treating the subject for a disease.

In some embodiments, the methods include treating, optionally in addition to one or more of monitoring, following-up with, advising, etc. a subject identified in any of the disclosed methods. Also disclosed are methods including treating, monitoring, following-up with, advising, etc. a subject for which a record of an identification from any of the disclosed methods has been made. For example, particular treatments, monitorings, follow-ups, advice, etc. can be used based on an identification and/or based on a record of an identification. For example, a subject identified as having a disease or condition with a high level of a particular component or characteristic (and/or a subject for which a record has been made of such an identification) can be treated with a therapy based on or directed to the high-level component or characteristic. Such treatments, monitorings, follow-ups, advice, etc. can be based, for example, directly on identifications, a record of such identifications, or a combination. Such treatments, monitorings, follow-ups, advice, etc. can be performed, for example, by the same individual or entity as, by a different individual or entity than, or a combination of the same individual or entity as and a different individual or entity than, the individual or entity that made the identifications and/or record of the identifications. The disclosed methods of treating, monitoring, following-up with, advising, etc. can be combined with any one or more other methods disclosed herein, and in particular, with any one or more steps of the disclosed methods of identification.

For example, in some embodiments, the methods include treating the subject for a disease when the serum sample includes at least a threshold value of free monoclonal immunoglobulin kappa light chains and/or a free immunoglobulin lambda light chain. For example, in some embodiments, the methods include treating the subject for a disease when an undiluted serum sample includes at least about 1.15 mg/L free monoclonal immunoglobulin kappa light chains and/or at least about 1.75 mg/L free immunoglobulin lambda light chain, and/or when an undiluted urine sample includes at least about 0.56 mg/L free monoclonal immunoglobulin kappa light chains and/or at least about 1.03 mg/L free immunoglobulin lambda light chain.

Neoplastic monoclonal gammopathies (NMG) include monoclonal gammopathy of undetermined significance (MGUS), asymptomatic or smoldering multiple myeloma (SMM) and multiple/plasma cell myeloma (MM) Of these only the malignant entity, MM, is treated in routine clinical care with antineoplastic drugs. By contrast the pre-malignant conditions of MGUS and SMM are usually observed, and treatment initiated when the lesions meet the criteria for MM (Fonseca, et al. Am. Soc. Clin. Oncol. Educ. Book. 2020; 40:1-7; Lonial, et al., J. Clin. Oncol. 2020; 38:1126-1137; Kim, et al., Cancers. 2020; 12:1223-1240). However, a physician is likely to recommend periodic checkups to monitor health, probably starting six months after diagnosis. If the subject is at high risk of MGUS or SMM developing into a more serious condition, the doctor may recommend more frequent checkups so that any progression can be diagnosed and treatment started as soon as possible. Doctors may watch for, and manage, signs and symptoms including bone pain, fatigue or weakness, unintentional weight loss, fever or night sweats, headache, dizziness, nerve pain, or changes in vision or hearing, bleeding, anemia or other blood abnormalities, swollen lymph nodes, liver or spleen. If the subject has osteoporosis, treatment may include a medication to increase bone density. Examples include alendronate (Fosamax), risedronate (Actonel, Atelvia), ibandronate (Boniva) and zoledronic acid (Reclast, Zometa).

Patients with SMM have even more abnormal plasma cells than patients with MGUS, so they are more likely to develop multiple myeloma. Through research and clinical trials, experts are exploring new treatment options for smoldering myeloma to reduce the risk of progression. For example, there are studies investigating treatment with monoclonal antibodies and vaccines. There are also clinical trials looking into immune-boosting steroids for patients with high-risk disease.

Multiple myeloma (and residual/minimal residual disease thereof) may be treated using one or more therapeutic approaches including, but not limited to:

Chemotherapy: Chemotherapy uses drugs to kill cancer cells. The drugs kill fast-growing cells, including myeloma cells. High doses of chemotherapy drugs are used before a bone marrow transplant. Examples of common chemotherapeutics traditionally used to treat MM include Cyclophosphamide (Cytoxan), Etoposide (VP-16), Doxorubicin (Adriamycin), Liposomal doxorubicin (Doxil), Melphalan, and Bendamustine (Treanda). Often one of these drugs is combined with other types of drugs like corticosteroids, proteasome inhibitors, thalidomide congeners and immuno-modulating agents.

Immunotherapy: Immunotherapy uses your immune system to fight cancer. The body's disease-fighting immune system may not attack the cancer because the cancer cells produce proteins that help them hide from the immune system cells. Immunotherapy works by interfering with that process. Exemplary agents include, but are not limited to, thalidomide (Thalomid), lenalidomide (Revlimid), and pomalidomide (Pomalyst). In patients, where the myeloma is in remission after either a stem cell transplant or initial treatment, lenalidomide may also be given for maintenance therapy to prolong the remission.

Corticosteroids: Corticosteroid medications regulate the immune system to control inflammation in the body. They are also active against myeloma cells. Corticosteroids help destroy myeloma cells and make chemotherapy more effective. The most common types used to treat myeloma are dexamethasone and prednisolone.

Targeted therapy: Targeted drug treatments focus on specific weaknesses present within cancer cells. By blocking these abnormalities, targeted drug treatments can cause cancer cells to die.

Bone marrow transplant: A bone marrow transplant, also known as a stem cell transplant, is a procedure to replace your diseased bone marrow with healthy bone marrow. In some embodiments, the stem cells are obtained from a donor, allogeneic donor. For example, in some embodiments, in treatment of multiple myeloma the patient's own stem cells, autologous donor, are used. Before a bone marrow transplant, blood-forming stem cells are collected from the subject's blood, a high dose of chemotherapy is used to destroy the diseased bone marrow, then the stem cells are infused into the body, where they travel to the bones and begin rebuilding the bone marrow.

Radiation therapy: Radiation therapy uses high-powered energy beams from sources such as X-rays or protons to kill cancer cells. It may be used to quickly shrink myeloma cells in a specific area—for instance, when a collection of abnormal plasma cells forms a tumor (plasmacytoma) that's causing pain or destroying a bone, or compressing spinal cord or other nerves.

Other treatments include, but are not limited to, proteasome inhibitors (e.g., bortezomib (Velcade), carfilzomib (Kyprolis), and ixazomib (Ninlaro)), monoclonal antibodies (e.g., anibodies against CD38 such as daratumumab (Darzalex), isatuximab (Sarclisa) and antibodies against SLAMF7 such as elotuzumab (Empliciti)), antibody-drug conjugates (e.g., Belantamab mafodotin-blmf (Blenrep)), nuclear export inhibitors (e.g., Selinexor (Xpovio)) and bisphosphonates (e.g., pamidronate (Aredia) and zoledronic acid (Zometa) and the drug denosumab (Xgeva, Prolia)) for bone disease.

Specific exemplary combination therapies include, but are not limited to:
Lenalidomide (or pomalidomide or thalidomide) and dexamethasone
Carfilzomib (or ixazomib or bortezomib), lenalidomide, and dexamethasone
Bortezomib (or carfilzomib), cyclophosphamide, and dexamethasone
Elotuzumab (or daratumumab), lenalidomide, and dexamethasone
Bortezomib, liposomal doxorubicin, and dexamethasone
Elotuzumab, bortezomib, and dexamethasone
Melphalan and prednisone (MP), with or without thalidomide or bortezomib
Vincristine, doxorubicin (Adriamycin), and dexamethasone (called VAD)
Dexamethasone, cyclophosphamide, etoposide, and cisplatin (called DCEP)
Dexamethasone, thalidomide, cisplatin, doxorubicin, cyclophosphamide, and etoposide (called DT-PACE), with or without bortezomib
Selinexor, bortezomib, dexamethasone

See, e.g., American Cancer Society website article entitled: “Drug Therapy for Multiple Myeloma.”

Blinatumomab, a bispecific T-cell engager (BiTE) associated with improved survival in relapsed or refractory acute lymphoblastic leukemia (ALL), was recently approved for treatment of minimal residual disease (MRD).

Any of the treatment methods can include autologous stem cell transplantation (ASCT).

In a more particular example, a treatment strategy may include a high-dose chemotherapy, (e.g., melphalan), with stem cell rescue and bio-therapies, such as lenalidomide, bortezomib, carfilzomib, daratumumab or radiation therapy.

B. Exemplary Methods

An exemplary method of identifying the presence of serum free monoclonal light chains in an undiluted biological sample, such as urine or serum from a subject by immunofixation electrophoresis (IFE) includes one or more of the steps of:
(i) depositing at least one aliquot portion of the undiluted sample on a deposit area of an electrophoretic gel plate having one anodic side and one cathodic side,
   wherein the sample deposit area is at a position of the gel plate allowing electrophoretic migration of the acidic/negatively charged protein content of the deposited sample towards the anodic side of the gel plate and neutral and positively charged proteins to the cathodic side;
(ii) electrophoresing the gel plate to obtain the undiluted protein separation profile;
(iii) contacting the electrophoresed gel with a solution including at least one capture antibody having specificity for free immunoglobulin light chain proteins or fragments thereof,
   wherein the contacting is under conditions that permit the formation of precipitated and/or detectable immunocomplexes between the capture antibody and free monoclonal immunoglobulin light chain proteins or fragments thereof within the protein separation profile;
(iv) removing unbound capture antibody by blotting the solution including at least one capture antibody by contacting the gel with blotting paper;
(v) contacting the gel with a wash solution including saline, and incubating the gel with the wash solution for at least one minute, preferably three minutes, then removing the wash solution, e.g., by blotting with filter papers;
(vi) repeating step (v) from one to ten times, inclusive; and
(vii) optionally, staining and/or quantitating the immunocomplexes formed in step (iii).

In some embodiments, the staining and/or quantitating the immunocomplexes in step (vii) includes drying the gel and staining the gel with a dye suitable for visualization of the precipitated immunocomplex and for quantitation, e.g., by densitometry scanning.

Preferably the capture antibody having specificity for free immunoglobulin light chain proteins or fragments thereof is a polyclonal antiserum.

In an exemplary form, a method of identifying the presence of free monoclonal light chains in an undiluted serum sample or other body fluid or extract of cells or tissue from a subject by immunofixation electrophoresis (FLC-UIFE), includes the steps of:
(i) depositing at least one aliquot portion of the undiluted serum sample on a deposit area of an electrophoretic gel plate having one anodic side and one cathodic side,
wherein the sample deposit area is at a position of the gel plate allowing electrophoretic migration of negatively charged/acidic proteins within the deposited undiluted serum sample towards the anodic side of the gel plate and migration of positively charged/basic proteins towards the cathodic end of the gel plate;
(ii) electrophoresing the gel plate to provide an undiluted serum protein separation profile;
(iii) contacting the electrophoresed gel with a solution comprising at least one capture antibody,
wherein the capture antibody has specificity for free immunoglobulin light chain proteins or fragments thereof,
wherein the contacting is under conditions that permit the formation of precipitated and/or detectable immunocomplexes between the capture antibody and free immunoglobulin light chain proteins or fragments thereof within the protein separation profile;
(iv) removing unbound capture antibody,
wherein the removing comprises blotting the solution comprising at least one capture antibody by contacting the gel with blotting paper;
(v) contacting the gel with a wash solution for at least one minute, preferably three minutes, then removing the wash solution;
(vi) optionally, repeating step (v) from one to ten times, inclusive; and
(vii) optionally, staining and/or quantitating the immunocomplexes formed in step (iii).

In an exemplary form, a method of identifying the presence of free monoclonal light chains in a concentrated urine sample from a subject by immunofixation electrophoresis (FLC-UIFE), includes the steps of
(i) concentrating a urine sample including proteins to provide a concentrated urine sample,
wherein the concentrating is effective to reduce the volume of the sample from about 5 fold to about 200-fold, inclusive;
(ii) depositing at least one aliquot portion of the concentrated urine sample on a deposit area of an electrophoretic gel plate having one anodic side and one cathodic side,
wherein the sample deposit area is at a position of the gel plate allowing electrophoretic migration of negatively charged/acidic proteins within the deposited concentrated urine sample towards the anodic side of the gel plate and migration of positively charged/basic proteins towards the cathodic end of the gel plate;
(iii) electrophoresing the gel plate to provide a protein separation profile of the concentrated urine sample;
(iv) contacting the electrophoresed gel with a solution including at least one capture antibody,
wherein the capture antibody has specificity for free immunoglobulin light chain proteins or fragments thereof, and
wherein the contacting is under conditions that permit formation of precipitated and/or detectable immunocomplexes between the capture antibody and free immunoglobulin light chain proteins or fragments thereof within the protein separation profile;
(v) removing unbound capture antibody,
wherein the removing comprises blotting the solution comprising at least one capture antibody by contacting the gel with blotting paper;
(vi) contacting the gel with a wash solution for at least one minute, preferably three minutes, then removing the wash solution;
(vii) optionally, repeating step (v) from one to ten times, inclusive; and
(viii) optionally, staining and/or quantitating the immunocomplexes formed in step (iv).

Exemplary apparatus that can be modified for conducting the methods is the Helena SIFE kit. In some embodiments, protein electrophoresis equipment from other vendors, e.g., Sebia is adapted to carry out the test by using undiluted serum, antisera to free light chains and the multiple wash steps described above, followed by staining for immunocomplexes.

C. Other Detection Methods

In some embodiments, the methods include repeating the methods, and/or comparing the results obtained by the methods with those obtained by one or more alternative methods for detecting free serum monoclonal light chains in the same or different sample from the same or different subject. Therefore, in some embodiments, the methods are carried together with, or prior to, or following one or more additional methods for identifying serum free monoclonal light chains in a sample. In certain embodiments, the methods test a urine sample from the patient to identify and quantify monoclonal light chains in urine. For example, in some embodiments, detection of monoclonal light chains in the urine supports the finding of serum analysis and may be more useful in patients with light chain monoclonal gammopathy. Exemplary alternative methods for detecting the presence of free serum monoclonal light chains in a biological sample, such as a serum or urine sample are known in the art and include, but are not limited to classical Serum protein electrophoresis (SPE) and classical Immunofixation electrophoresis (IFE), Capillary Electrophoresis (CE) is also used for electrophoretic analysis of the immunoglobulins contained in a biological sample. A particular adaptation of CE relying on an immuno-displacement step, is used for identifying monoclonal proteins which may be present in an analyzed biological sample.

Capillary Electrophoresis immuno-displacement may use a chemically modified antibody that despite such modification retains its ability to bind monoclonal proteins. This chemical modification provides additional negative charge to the antibodies to allow antibodies and their complexes to move out of the gamma zone, or out of the serum protein pattern during the electrophoretic migration. For immuno-displacement CE, the sample is necessarily pre-incubated with a specific modified antiserum (antibody), before subjecting the resulting mixture to capillary electrophoresis process. Disappearance or not of a peak from gamma zone during the migration with this specific modified antiserum allows, in simple cases, the classification and typing of the sample. However, these methods are generally less sensitive and less effective than the described methods for FLC-Modified SIFE.

In a preferred embodiment, the alternative system to identify the presence and quantity of serum free monoclonal immunoglobulin light chains is Mass Spectrometric analysis following nanobody mediated concentration of immunoglobulins based matrix desorption time of flight analysis (MASS-FIX/MALDI). As demonstrated in the Examples, FLC-Modified SIFE, using undiluted serum sample for electrophoresis, using antisera to free light chains and multiple wash steps is more sensitive and specific than other methods, such as MASS-FIX MALDI. (see also, Wilhite et al., Pract Lab Med 2021; 27:e00256).

D. Subjects

The methods can include one or more steps of identifying a subject in need of the methods. For example, in some embodiments, the methods include identifying a subject and obtaining a biological sample from the subject. In preferred embodiments, the methods include one or more steps for providing an undiluted serum, and/or urine sample, preferably a concentrated urine sample, from a subject identified as being in need of assessing for the presence of free monoclonal IgL light chains or monoclonal IgK light chains. Typically, the subject is a human subject, such as a human subject identified as having, or suspected of having a neoplastic monoclonal gammopathy (NMG) such as monoclonal gammopathy of undetermined significance (MGUS), asymptomatic or smoldering multiple myeloma (SMM) and multiple/plasma cell myeloma (MM), or another disease such as AIDS, Chronic lymphocytic leukemia, Non-Hodgkin Lymphoma, particularly Splenic marginal zone lymphoma and Lymphoplasmocytic lymphoma, Hepatitis C, Connective tissue disease such as lupus, Immunosuppression following organ transplantation, Waldenstrom macroglobulinemia, Guillain-Barre syndrome, polyneuropathy, amyloidosis or Tempi syndrome. In preferred embodiments the subject is a human patient having or suspected of having a light chain multiple myeloma (LCMM) or light-chain-predominant MM (LCPMM).

In some embodiments, the subject is receiving or has previously received treatment for a Neoplastic monoclonal gammopathy (NMG). An exemplary treatment is Autologous stem cell transplantation (ASCT). In some embodiments, the subject has, or is suspected as having residual/minimal residual disease (MRD). In some embodiments, the subject is receiving or has previously received treatment using one or more monoclonal antibody therapeutics.

E. Diagnostic Systems

Diagnostic systems that include the methods are provided. In some embodiments, the methods determine a disease or disorder state of a subject based on the evaluation of the amount of free monoclonal immunoglobulin light chain proteins within a sample from the subject.

In some embodiments, the methods include repeating or reproducing the steps of the method, for example to provide multiple results. Therefore, in some embodiments, the methods include compiling or comparing two or more results and comparing results from other body fluids, such as urine, or from bone marrow cells or cells from a localized tumor of plasma cells, i.e., plasmacytoma. In some embodiments, the methods include compiling and/or comparing the results obtained from a sample with those obtained from a different sample, including a different fluid such as urine. In some embodiments, the methods include compiling and/or comparing the results obtained from a first sample with those obtained from a second or further sample and/or from a different fluid. The different samples are typically from the same subject. The different samples can be from the same or different fluid, and/or obtained at the same or different location, at the same or different times relative to one another. In some embodiments, the methods include compiling and/or comparing the results obtained from a urine sample with those obtained from a serum sample from the same subject, for example at the same or different times. In some embodiments, the methods determine a disease or disorder state of a subject based on the evaluation of the amount of free monoclonal immunoglobulin light chain proteins within a urine sample, a serum sample, or both a urine sample and serum sample from the same subject. In some embodiments, the methods determine a disease or disorder state of a subject based on the evaluation of the amount of free monoclonal immunoglobulin light chain proteins within a urine sample, a serum sample, or both a urine sample and serum sample from the same subject by comparing with the amount of free monoclonal immunoglobulin light chain proteins within one or more control samples, such as a control urine sample and/or a control serum sample having a known amount of free monoclonal immunoglobulin light chain proteins.

The disclosed methods can further involve the use of a computer system to compare levels of the one or more of the disclosed biomarkers to control values. For example, the computer system can use an algorithm to compare levels of two or more biomarkers and provide a score representing the risk of disease onset based on detected differences.

This algorithm can in some embodiments weigh multiple parameters. For example, in some embodiments, the algorithm gives weight depending on which biomarker demonstrates differences, e.g., more weight to differences in IgK and/or IgL levels over other biomarkers. In some embodiments, the algorithm weighs the extent of elevation or decrease in biomarkers levels compared to the control. For example, a 50% reduction in biomarker levels may be weighted more than a 20% reduction in the same biomarker. In other embodiments, the algorithm gives weight to differences in biomarker levels for a combination of biomarkers.

Therefore, also provided is an apparatus for use in detecting MM, or any other disease or disorder associated with the increased or abnormal presence of free monoclonal immunoglobulin light chain in a subject that includes an input means for entering biomarker level values from a sample of the subject, a processor means for comparing the values to control values, an algorithm for giving weight to specified parameters, and an output means for giving a score representing the risk of disease onset.

### III. Compositions of Protein Biomarkers for Immunoassays

Compositions and methods are provided for identifying one or more determinants of diseases or disorders in an undiluted sample from a subject are provided. In an exemplary embodiment, the compositions allow the screening of Neoplastic monoclonal gammopathies, such as of multiple/plasma cell myeloma (MM) in a subject.

The disclosed compositions include biomarkers that can be used to determine a subject's diagnosis or prognosis. For example, the disclosed biomarkers can in some embodiments be used to determine whether the subject has residual/minimal residual disease of lymphoplasmic disorders. In other embodiments, the biomarkers can be used to determine whether the subject is at risk of developing multiple myeloma, for example, if a subject is determined as having monoclonal gammopathy of undetermined significance (MGUS), or asymptomatic or smoldering multiple myeloma (SMM). For example, in some embodiments, the presence and/or amount of biomarkers determine the course or progression of disease in a subject who is at risk of progressing from SMM to MM.

A. Free Monoclonal Immunoglobulin Light and Heavy Chains

The described methods detect free monoclonal immunoglobulin light chains in an undiluted biological sample, such as an undiluted serum, urine sample, or other body fluids and cell extracts. In an exemplary embodiment, the method is applied to the detection of free monoclonal heavy chains by using antisera specific for heavy chains. “Light chains” are proteins made by plasma cells, a type of white blood cell, also known as “Bence Jones proteins” and high levels of these proteins in blood indicate a B cell-related neoplasms such as plasma cell dyscrasia, including MM. There are 2 types of light chains, termed kappa (κ) and lambda (λ). The term “monoclonal” refers to an immunoglobulin belonging to a single immunoglobulin class, or a fragment thereof, as defined by heavy and/or light chain immunoglobulin isotypes and produced by a single specific clone of B cells in association with a pathological context. In some forms, monoclonal immunoglobulins are intact immunoglobulin molecules composed of one type of heavy chain and one type of light chains. Such molecules are the commonest monoclonal immunoglobulins and are produced by a single clone of lympho-plasmacytic cells. About 85% of the MM produce intact immunoglobulins. However, such tumors usually also produce excess free monoclonal light chains. Another common type of monoclonal immunoglobulin is a light chain of only one type produced by a single clone of lympho-plasmacytic cells. Such lesions account for about 15% of MM.

Monoclonal gammopathies are characterized by the increased production of monoclonal protein (MP), following an abnormal proliferation of a single plasma-cell clone. Immunoglobulins are produced in the endoplasmic reticulum of B-cells as tetramers made up of two identical heavy chains (HC) of G, A, M, D, or E class, and two identical light chains (LC). The latter exist as two isotypes, namely kappa (κ) and lambda (k), linked to heavy chains through disulfide bonds and non-covalent interactions. The genes encoding for HC are present on chromosome 14, whereas those encoding for κ and λ LC are on chromosomes 2 and 22, respectively. Both κ and λ LC are synthesized in excess compared to the HC counterpart. The LC excess secreted in blood represents the serum Free Light Chains (sFLC), Both κ and λ LC may exist singly or as dimers bound to each other by either covalent (disulfide) or non-covalent links, although k FLC have a stronger tendency to dimerization/oligomerization than κ FLC.

Monoclonal proteins may be either intact immunoglobulins, IgG, IgM, IgA, or more rarely IgE and IgD, a combination of both intact immunoglobulins and free light chains, free light chains only, or heavy chains only. The most common plasma cell disorders include monoclonal gammopathy of undetermined significance (MGUS), smoldering multiple myeloma (SMM) multiple myeloma (MM), amyloidosis (AL), B-cell lymphomas including lymphoplasmacytic lymphoma/Waldenstrom's macroglobulinemia (WM), and Polyneuropathy-Organomegaly-Endocrinopathy-Monoclonal Protein-Skin changes (POEMS) syndrome. Approximately 80 to 85% of myelomas secrete intact immunoglobulins, 13 to 15% only light chains, and 1 to 2% are non- or oligo-secretory myelomas. Laboratory tests for the diagnosis of monoclonal gammopathies include serum protein electrophoresis (SPE/SPEP), serum protein immunofixation electrophoresis (SIFE), urine protein electrophoresis (UPEP), urine protein immunofixation electrophoresis (UIFE), and quantification of serum free κ and λ light chains (SFLC), such as Freelite (The Binding Site), N-Latex FLC (Siemens), Seralite (Abingdon Health), Sebia FLC (Sebia) and Diazyme.

B. Undiluted Sample

Methods to determine the presence and quantity of free IgK and free IgL have been established. The methods preferably utilize undiluted biological samples. Therefore, undiluted samples are provided. The undiluted sample can be a biological or environmental sample. Preferably, the undiluted sample is a biological sample, for example, all or part of a sample obtained from a subject.

The term “undiluted” refers to a sample that contains an approximately physiological or “natural” level of components. Concentrated samples are a form of undiluted sample. The term “concentrated” refers to a sample that contains an approximately greater concentration of one or more components than the physiological or “natural” concentration of components. Therefore, in some embodiments, the undiluted sample is a concentrated undiluted sample. In other forms, the undiluted sample is a non-concentrated undiluted sample.

Biological samples include any bodily fluid, as well as tissues and/or cells obtained from a subject. Techniques for obtaining a biological sample from a subject are well known in the art. In some embodiments, the analyzed biological samples are selected among: organs, tissue, bodily fluids, and cells. Where the biological sample is a bodily fluid, the fluid can be selected from blood, serum, plasma, urine, sputum, saliva, stool, spinal fluid, cerebral spinal fluid, lymph fluid, skin secretions, respiratory secretions, intestinal secretions, genitourinary tract secretions, tears, and milk, etc. The organs include, e.g., the adrenal glands, bladder, bones, brain, breasts, cervix, esophagus, eyes, gall bladder, genitals, heart, kidneys, large intestine, liver, lungs, lymph nodes, ovaries, pancreas, pituitary gland, prostate, salivary glands, skeletal muscles, skin, small intestine, spinal cord, spleen, stomach, thymus gland, trachea, thyroid, testes, ureters, and urethra. Tissues include, e.g., epithelial, connective, nervous, and muscle tissue.

Preferred biological samples include serum and urine. In some embodiments the sample is a serum sample, for example, a serum sample obtained from a sample of blood. In other embodiments, the sample is a urine sample. Typically, the undiluted sample is provided in the form of a liquid. In some embodiments, the undiluted sample is derived from a solid, such as a frozen liquid or a dried powder, such as a lyophilized powder. Therefore, in some embodiments, providing an undiluted sample includes one or more steps of preparing a liquid sample from a solid sample, for example, by thawing or dissolving in a suitable excipient, such as water or saline. In other forms, the sample is a concentrated sample. When the sample is prepared by dissolving in a suitable excipient, the total volume of the dissolved sample should not exceed that of the sample prior to concentrating, drying or lyophilization. In some embodiments, samples are created by lysing cells and tissues.

C. Controls

In some embodiments, the methods include one or more control samples, such as a positive control sample, for example, containing a biological sample, such as serum or urine, including a known amount of a control protein. An exemplary control protein is a single light chain protein of a monoclonal immunoglobulin, such as a kappa or lambda light chain protein. Another exemplary control specimen is a serum containing a monoclonal protein such as an intact monoclonal immunoglobulin of known type and concentration. Typically, the type and quantity of the control proteins are known, and the positions of the control proteins within a separation profile obtained by electrophoresis of the control sample are also known. Therefore, in some embodiments, the methods include depositing one or more positive controls onto the electrophoresis plate together with an aliquot of the biological sample, being tested and has unknown content of monoclonal immunoglobulins, whereas the positive control includes one or more target proteins that may or may not be present within the biological sample. The control and unknown or test specimen are applied to the gel in different areas/spots/wells.

In some embodiments, the methods include one or more negative control or reference samples that do not contain monoclonal immunoglobulins. A control may also include a sample on which no antiserum is applied, or on which only one or more exogenous proteins are applied. In other embodiments, a negative control includes a biological sample obtained from a healthy subject, such as a subject who does not have a disease or disorder that results in the presence of a target protein within the biological sample, or who has been identified as having a smaller or healthy amount thereof.

In some embodiments, a negative and a positive control allow the typing of each monoclonal band that is visible on the gel, by comparison with one or more negative and/or positive controls. Immunoglobulins are generally formed from heavy chains (2 heavy chains) and light chains (2 light chains). Five heavy chain isotypes (M, G, A, D, E″ isotypic classes) and two light chain isotypes (kappa and lambda isotypic types) have been identified in that four-chain structure. Therefore, in some embodiments, a negative or a positive control includes one or more of a known amount of an immunoglobulin formed from heavy chains (2 heavy chains) and light chains (2 light chains) of one or more of the five heavy chain isotypes (M, G, A, D, E″ isotypic classes) and/or two light chain isotypes (kappa and lambda isotypic types). In some embodiments, a negative or a positive control include an exogenous or endogenous antibody or immunoglobulin, such as a therapeutic monoclonal antibody, or endogenous immunoglobulins, including endogenous monoclonal immunoglobulin or polyclonal antiserum or component thereof.

In some embodiments, the control includes at least about 1.15 mg/L free monoclonal immunoglobulin kappa light chains. In some embodiments, the control includes at least about 1.75 mg/L free monoclonal immunoglobulin lambda light chain. In some embodiments, the control includes at least about 1.15 mg/L free monoclonal immunoglobulin kappa light chain and at least about 1.75 mg/L free monoclonal immunoglobulin lambda light chain.

D. Capture Agents

The methods determine the presence and quantity of IgK and IgL in an undiluted sample using immunofixation by binding of the IgK and IgL to a specific capture agent.

1. Capture Antibodies

A preferred capture agent is an immunoglobulin (“capture antibody”) that selectively and specifically binds to free immunoglobulin light chains in the context of the immunoassay, such as kappa light chains, or lambda light chains. The capture antibodies bind to normal polyclonal light chains as well as to abnormal monoclonal light chains associated with NMG, including MM.

Preferred capture antibodies include polyclonal antisera, such as polyclonal antisera specific to free immunoglobulin light chains. In some embodiments the capture agent is a polyclonal antibody that specifically binds to free immunoglobulin kappa light chains (IgK). In other embodiments the capture agent is a polyclonal antibody that specifically bind to free immunoglobulin lambda light chains (IgL). In some embodiments the capture agent is a mixture of polyclonal antibodies that specifically bind to free immunoglobulin kappa light chains (IgK) and to free immunoglobulin lambda light chains (IgL). antisera to IgL, or antisera to both IgK and IgL.

Antisera specific for free light chains are generally raised to epitopes on light chains that are hidden in the intact immunoglobulin. Such hidden epitopes specific for both kappa and lambda light chains have been identified and polyclonal antisera specific to free light chains produced accordingly. Monoclonal antibodies specific for free light chains have also been produced for measuring the quantities of free light chains in body fluids. However, polyclonal antibodies are typically preferred for precipitation reactions such as those employed in immunofixation electrophoresis.

Specificity of reactivity of antisera to free light chains can be verified by staining sera from patients with known immunotypes of monoclonal gammopathies.

Capture antibodies specific to IgK or IgL are available from multiple commercial sources, and include Rabbit polyclonal antisera to kappa and lambda free light chains were procured from SEBIA Inc. (Norcross, GA, USA.)(Catalogue Nos: 4601 and 46011), Helena Laboratories (Beaumont TX) (Catalogue No. 9412 and 9413), Agilent (Santa Clara, CA) (Catalogue No. A0100 and A0101), and mouse monoclonals, e.g., Gentaur Antibodies (San Jose, CA) (Product number 065405F03, and Antibody specificity Ig lambda chain C region (Lambda light chain); Product number 118411C12H, Antibody specificity Human kappa light chain protein (Bence Jones protein)), eBioscience™ Lambda light chain Antibody (12-9990-42) clone 1-155-2; Anti-Abcam catalogue number ab1944—Lambda Free Light Chain antibody [3D12], the murine IgG1 s anti-κ (Fκ-C8) and anti-λ (Fλ-G9) FLC mAbs (Abe, et al. Clin Exp Immunol. 111:457-4621998), and HRP-conjugated goat F(ab′)2 anti-human κ or λ antibodies (Biosource, Camarillo, CA) (e.g., diluted 1:1000 in saline). See also Davern, et al. Am J Gun Pathol. 2008 November; 130(5):702-11. doi: PMID: 18854262; PMCID: PMC2620173.

2. Dyes and Labels

In some embodiments, the capture agents are labelled capture agents that enhance the detection of the immunocomplexes formed by the capture agents and the target proteins. For example, in some embodiments the capture antibodies are labelled capture antibodies. Labelling can be direct or indirect. A label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known as fluorochromes and fluorophores) and enzymes that react with colorometric substrates (e.g., horseradish peroxidase, and alkaline phosphatase). The use of fluorescent dyes is generally preferred in the practice of the disclosure as they can be detected at very low amounts. Furthermore, in the case where multiple antigens are reacted with a single array, each capture antibody can be labeled with a distinct fluorescent compound for simultaneous detection. Labeled spots on the array are generally detected visually/photographically, e.g., by illuminating with light of suitable wavelength, or using a fluorimeter, with the presence of a signal indicating an antigen bound to a specific antibody.

A modifier unit such as a radionuclide can be incorporated into or attached directly to any of the disclosed compositions by halogenation. In another aspect, the radionuclide can be attached to a linking group or bound by a chelating group, which is then attached to the compound directly or by means of a linker. Radiolabeling techniques such as these are routinely used in the radiopharmaceutical industry.

Labeling can be either direct or indirect. In direct labeling, the detecting antibody (the capture antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avidin pair. In this mode, the detecting antibody or detecting molecule should include the other member of the pair.

Other modes of indirect labeling include the detection of primary immune complexes by a two-step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

In some embodiments, the electrophoresed gel including the immunocomplexes is stained using one or more dyes or stains. An exemplary dye is a mixture of Coomassie blue and Amido Black stain. Depending on the reagents used as capture agent and moieties linked primary antibody or secondary antibody, other detection methods include, fluorescence, enzyme reaction and visual dye or metal (e.g., Gold) detection.

### IV. Kits

Kits suitable for carrying out the disclosed methods are also provided. Typically, the kits include reagents including capture antisera specific to free light chains within undiluted serum, and/or concentrated urine, and for conducting multiple wash steps according to the described methods. In some embodiments, the kits include one or more of capture antibody(ies) specific for intact immunoglobulins IgG, IgA and IgM and light chains, though the antisera are not specific for free light chains and bind to light chains linked to heavy chains as well as free light chains; electrophoretic gels; positive and negative control samples including monoclonal immunoglobulins or no monoclonal immunoglobulin respectively. Other materials in the kits include blotting paper; wash solution; fixative solution; Gel stain; positive control samples including a known amount and type of monoclonal immunoglobulin.

The methods will be better understood in view of the following paragraphs:

1. A method of detecting free monoclonal light chains in serum including immunofixation electrophoresis (FLC-Modified SIFE),
wherein the FLC-Modified SIFE includes selectively labeling only free immunoglobulin light chain proteins, and
optionally quantifying the labelled free monoclonal immunoglobulin light chain proteins.

2. A method of identifying a subject as having a disease or disorder associated with free monoclonal light chains, or as being at risk of having a disease or disorder associated with free monoclonal light chains, including detecting free monoclonal light chains according to the method of paragraph 1.

3. The method of paragraph 1 or 2, wherein the amount of free monoclonal immunoglobulin light chain that is detected in the undiluted sample is 1.75 mg/L or more.

4. The method of any one of paragraphs 1 to 3, wherein selectively labeling includes contacting the undiluted serum protein separation profile with an antibody specific for free immunoglobulin light chain proteins,
wherein the contacting occurs under conditions that permit binding of the free light chains with the antibody.

5. The method of any one of paragraphs 1 to 4, wherein the FLC-Modified SIFE includes
(a) depositing at least one aliquot portion of the undiluted sample on a deposit area of an electrophoretic gel plate having one anodic side and one cathodic side,
   wherein the sample deposit area is at a position of the gel plate allowing electrophoretic migration of the acidic protein content of the deposited undiluted sample towards the anodic side of the gel plate and migration of positively charged/basic, proteins towards the cathodic end of the gel plate;
(b) electrophoresing the gel plate to obtain the undiluted serum protein separation profile;
(c) applying at least one capture antibody to the electrophoresed gel and permitting its reaction to allow the formation of precipitated and/or detectable immunocomplexes,
   wherein the capture antibody specifically binds to free immunoglobulin light chain proteins or fragments thereof.
(d) removing unbound capture antibody and excess proteins; and
(e) optionally, staining and/or quantitating the immunocomplexes formed in step (c).

6. The method of paragraph 5, wherein the capture antibody is a polyclonal antibody.

7. The method of any one of paragraphs 1-6, wherein the sample is selected from a serum sample and/or a urine sample.

8. The method of any one of paragraphs 1-7, wherein the sample is an undiluted serum sample or a concentrated urine sample.

9. The method of any one of paragraphs 5-8, wherein removal of unbound capture antibody after formation of the precipitated and/or detectable immunocomplexes in (d) includes blotting the gel to remove unbound capture antibody and incubating the gel in a wash solution.

10. The method of paragraph 9, wherein the blotting includes contacting the gel with blotting filter paper,
optionally wherein the incubation includes overlaying the gel with blotting filter paper, saturating the paper with a wash solution, incubating the gel with the filter paper and the wash solution.

11. The method of paragraph 9 or 10, wherein the wash solution includes saline and wherein the incubation time is from about one minute to about five minutes, inclusive, preferably three minutes.

12. The method of paragraph 11, wherein the washing is repeated two or more times.

13. The method of any one of paragraphs 5-12, wherein at least one aliquot portion of the undiluted sample is deposited on the gel plate as a reference which is not submitted to step (c) but is instead contacted with a fixative solution rather than with capture antibody(ies),
wherein steps (a), (b), (d) and optionally (e) remain the same.

14. The method of any one of paragraphs 5 to 13, wherein six aliquot portions of the undiluted or diluted sample are deposited on the gel plate in step (a), including a reference aliquot portion and three aliquot portions that are respectively contacted in step (c) with capture antibodies specific to Immunoglobulin G (IgG), Immunoglobulin A (lgA), and Immunoglobulin M (IgM), respectively, optionally further including staining one or more additional aliquot portion for total proteins.

15. The method of any one of paragraphs 5-14, wherein the detection of free monoclonal immunoglobulin light chain proteins is compared to one or more control samples,
wherein a protein separation profile of the control samples is produced by electrophoretic migration of the protein content of the control samples, and
wherein the control samples include one or more of a negative control, including no free monoclonal immunoglobulin light chain, and/or a positive control including a known concentration of one or more free monoclonal immunoglobulin light chain, or fragments thereof.

16. The method of any one of paragraphs 5-15, wherein one capture antibody is a polyclonal antibody that specifically binds to free human immunoglobulin kappa light chain.

17. The method of any one of paragraphs 5-16, wherein one capture antibody is a polyclonal antibody that specifically binds to free human immunoglobulin lambda light chain.

18. The method of any one of paragraphs 5-17, wherein the capture antibody is a polyclonal antibody specific for free human immunoglobulin kappa light chain, or for free human immunoglobulin lambda light chain.

19 The method according to any one of paragraphs 5 to 18, further including one or more steps of
(f) analyzing and/or interpreting the FLC-Modified SIFE results and/or concluding about the health status of the subject.

20. The method of paragraph 19, further including
(g) treating the subject for a disease when the sample contains monoclonal immunoglobulins and meets one or more additional criteria for the diagnosis of MM.
optionally wherein the treatment includes chemotherapy, immunotherapy, corticosteroids, targeted therapy, radiation therapy, proteasome inhibition, monoclonal antibodies against CD38 and/or SLAM7, antibody-drug conjugate therapy, nuclear export inhibition, bisphosphonate treatment for bone disease, CAR T cell therapy, autologous stem cell transplantation (ASCT), or a combination thereof.

21. The method of any one of paragraphs 1 to 20, wherein the disease or disorder associated with free monoclonal light chains is selected from the group including monoclonal gammopathy of undetermined significance (MGUS), asymptomatic or smoldering multiple myeloma (SMM), multiple/plasma cell myeloma (MM), HIV/AIDS, Chronic lymphocytic leukemia, Non-Hodgkin Lymphoma, particularly Splenic marginal zone lymphoma and Lymphoplasmacytic lymphoma, Hepatitis C, Connective tissue disease such as lupus, Immunosuppression following organ transplantation, Waldenstrom macroglobulinemia, Guillain-Barre syndrome, polyneuropathy, amyloidosis or Tempi syndrome.

22. The method of any one of paragraphs 1 to 21, wherein the disease or disorder associated with free monoclonal light chains is a conventional MM, light-chain-predominant multiple/plasma cell myeloma (LCPMM) or light chain myeloma/LCMM.

23. The method of any one of paragraphs 1 to 22 wherein the subject has previously been treated for a disease or disorder associated with free monoclonal light chains selected from the group including monoclonal gammopathy of undetermined significance (MGUS), asymptomatic or smoldering multiple myeloma (SMM), multiple/plasma cell myeloma (MM), HIV/AIDS, Chronic lymphocytic leukemia, Non-Hodgkin Lymphoma, particularly Splenic marginal zone lymphoma and Lymphoplasmacytic lymphoma, Hepatitis C, Connective tissue disease such as lupus, Immunosuppression following organ transplantation, Waldenstrom macroglobulinemia, Guillain-Barre syndrome, polyneuropathy, amyloidosis or Tempi syndrome.

24. The method of any one of paragraphs 1-23, wherein the subject has received, or is receiving treatment for a Neoplastic monoclonal gammopathy (NMG).

25. The method of any one of paragraphs 1 to 24, wherein the detection of free monoclonal kappa light chains at any concentration indicates the subject has residual/minimal residual disease (MRD).

26. The method of any one of paragraphs 1 to 25, wherein the detection of free monoclonal kappa light chains at any concentration indicates the subject has residual/minimal residual disease (MRD).

27. The method of any one of paragraphs 1-26, wherein the subject has received, or is receiving treatment using one or more monoclonal antibody therapeutics.

28. The method of any one of paragraphs 1-27, wherein the subject has previously been screened for the presence of monoclonal immunoglobulins in a biological sample by another technique, and wherein the result was previously found to be negative.

29. A method of identifying the presence of serum free monoclonal light chains in an undiluted serum sample, urine or other body fluid or extract of cells or tissue from a subject by immunofixation electrophoresis (FLC Modified SIFE), including
(i) depositing at least one aliquot portion of the undiluted serum sample on a deposit area of an electrophoretic gel plate having one anodic side and one cathodic side,
   wherein the sample deposit area is at a position of the gel plate allowing electrophoretic migration of the acidic protein content of the deposited undiluted serum sample towards the anodic side of the gel plate and migration of positively charged/basic proteins towards the cathodic end of the gel plate;
(ii) electrophoresing the gel plate to obtain the undiluted serum protein separation profile;
(iii) contacting the electrophoresed gel with a solution including at least one capture antibody having specificity for free immunoglobulin light chain proteins or fragments thereof,
   wherein the contacting is under conditions that permit the formation of precipitated and/or detectable immunocomplexes between the capture antibody and free immunoglobulin light chain proteins or fragments thereof within the protein separation profile;
(iv) removing unbound capture antibody by blotting the solution including at least one capture antibody by contacting the gel with blotting paper;
(v) contacting the gel with a wash solution including saline, and incubating the gel with the wash solution for at least one minute, preferably three minutes, then removing the wash solution;
(vi) repeating step (v) from one to ten times, inclusive; and
(vii) optionally, staining and/or quantitating the immunocomplexes formed in step (iii).

30. A method of identifying the presence of free monoclonal light chains in a urine sample from a subject by immunofixation electrophoresis (FLC-UIFE), including
(i) concentrating the proteins within a urine sample to achieve a 5 to 200-fold reduction in volume.
(ii) depositing at least one aliquot portion of the concentrated urine sample on a deposit area of an electrophoretic gel plate having one anodic side and one cathodic side,
   wherein the sample deposit area is at a position of the gel plate allowing electrophoretic migration of the acidic protein content of the deposited concentrated urine sample towards the anodic side of the gel plate and migration of positively charged/basic proteins towards the cathodic end of the gel plate;
(iii) electrophoresing the gel plate to obtain the protein separation profile of the concentrated urine sample;
(iv) contacting the electrophoresed gel with a solution including at least one capture antibody having specificity for free immunoglobulin light chain proteins or fragments thereof,
   wherein the contacting is under conditions that permit the formation of precipitated and/or detectable immunocomplexes between the capture antibody and free immunoglobulin light chain proteins or fragments thereof within the protein separation profile;
(v) removing unbound capture antibody by blotting the solution including at least one capture antibody by contacting the gel with blotting paper;
(vi) contacting the gel with a wash solution including saline, and incubating the gel with the wash solution for at least one minute, preferably three minutes, then removing the wash solution;
(vii) repeating step (v) from one to ten times, inclusive; and
(viii) optionally, staining and/or quantitating the immunocomplexes formed in step (iv).

31. The method of paragraph 29 or 30, wherein contacting the gel with a wash solution includes:
(I) contacting the gel with a saline wash solution;
(II) incubating the gel in the wash solution for 3 min;
(III) contacting the gel with blotting paper to remove the wash solution;
(IV) repeating steps (I-III) twice or more times;
(V) contacting the gel with blotting paper filter by overlaying the gel with the paper and saturating the paper with saline wash solution;
(VI) incubating the gel in the wash solution for 3 min;
(VII) removing the filter paper and contacting the gel with more blotting paper filters to remove the wash solution; and
(VIII) repeating steps (V-VI) twice or more times.

32. The method of any one of paragraphs 29-31, wherein the staining and/or quantitating the immunocomplexes in step (iv) includes drying the gel and staining the gel with a dye suitable for quantitation and visual examination.

33. A kit suitable for carrying out a method according to any one of paragraphs 1 to 32, including one or more of
(i) capture antibody(ies) specific for free immunoglobulin light chain;
(ii) electrophoretic gels;
(iii) negative control samples including no free monoclonal immunoglobulin light chain;
(iv) blotting paper;
(v) wash solution;
(vi) fixative solution;
(v) gel stain;
(vii) positive control samples including a known amount and type of free immunoglobulin light chain; and
(viii) apparatus for obtaining a biological sample from a subject.

### EXAMPLES

### Example 1: Detection of Free Monoclonal Light Chains in Serum by Modified Immunofixation Electrophoresis (FLC-Modified SIFE) with Antisera Against Free Light Chains

To ascertain the nature of multiple bands on SIFE in a patient with light-chain-predominant Multiple Myeloma (LCPMM), polyclonal antisera to free light chains in SIFE was assessed as a means to enhance the sensitivity of detection of monoclonal SFLC in serum. The test could serve to detect residual/minimal residual disease (MRD).

Methods

The investigation was carried out at a 480 bed, tertiary care hospital, affiliated with a medical school in the Southeastern USA. The medical center offers matched unrelated donor, allogeneic, umbilical cord blood, and autologous stem cell transplants for hematologic malignancies along with providing other tertiary care and oncology services to the region. The study protocol was reviewed and approved by the Augusta. University institutional review hoard (Protocol It 657783).

Specimens submitted for SPEP/SIFE were analyzed and results reported by the standard clinical methods as has been described previously. Quantification of SFLC was conducted by using kits procured from The Binding Site and assayed with an Optilite analyzer. Rabbit polyclonal antisera to kappa and lambda free light chains were procured from SEBIA Inc. (Norcross, GA, USA.) Residual clinical serum samples were assessed for monoclonal SFLC by a Modified SIFE procedure, as described below: (The Modified SIFE will be designated FLC-Modified SIFE).

FLC-Modified SIFE

Undiluted serum samples were applied to SIFE gels procured from Helena, using Helena SPIFE touch equipment. (Helena Laboratories, Beaumont, TX). Some specimens were examined by triple application of the undiluted serum inoculum. This was done primarily to confirm negative results obtained via single application.

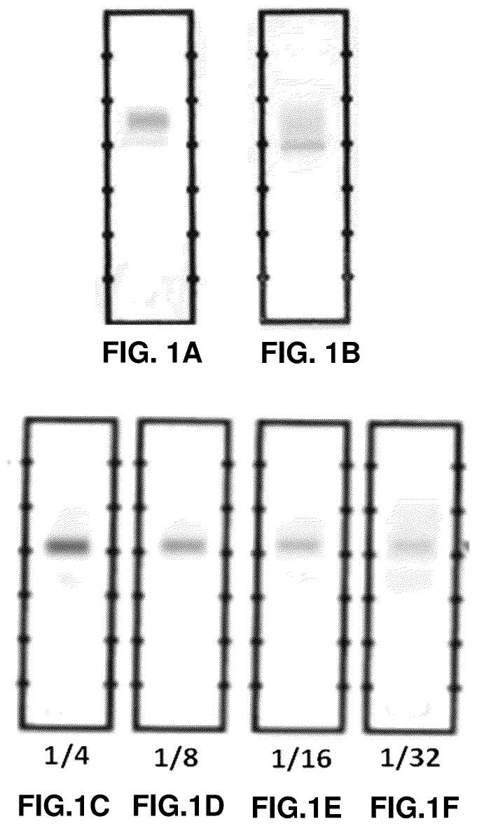

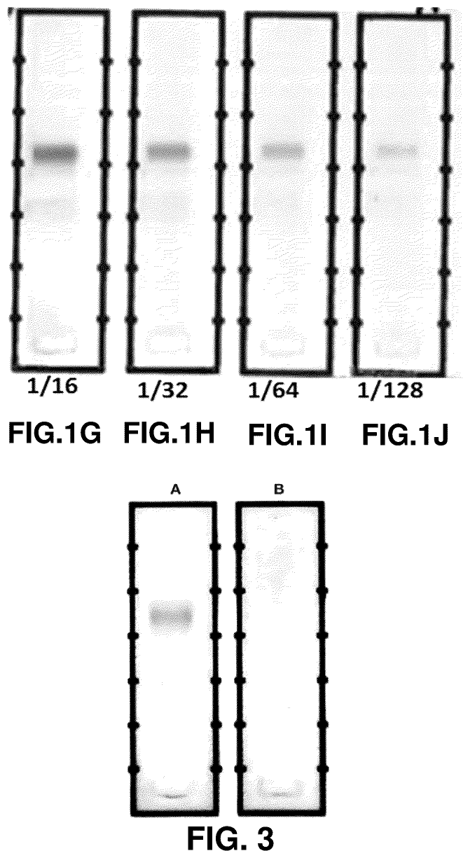

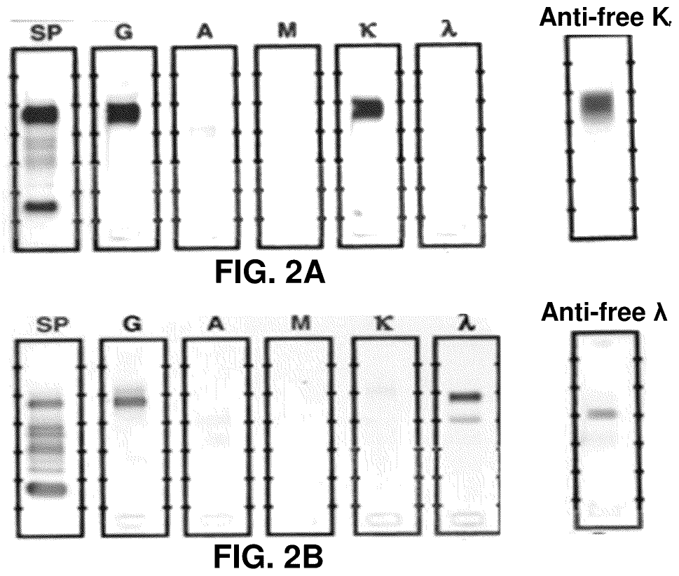

Electrophoresis was carried out according to the program and instructions provided according to the manufacturer. Following electrophoresis, 50 μL of antisera to free kappa or lambda light chains were applied to selective electrophoretic slots and incubated according to the manufacturer protocol. Following a standard incubation period with antiserum, the antiserum was blotted with SIFE filters in SIFE kits (Helena). Following blotting of excess antisera, 504, of saline was added to the antibody slots and incubated for 3 min followed by blotting the excess solution as was done for the initial antiserum. This process was repeated two more times. The gel was subjected to blotting with two filter papers, filter “C” and filter “D”, according to the manufacturer's protocol. The gel was overlaid with fresh filter “C” and the filter paper was flooded with saline and incubated for 3 min. The filter paper was removed followed by blotting with filters “C” and “D”. Application of filter C, flooding with saline and blotting with filters C and D was repeated twice more. The blotted gel was dried and stained according to the manufacturer's protocol. The stained gel was evaluated visually (FIGS. 1A-1C and 2A-2B).

Specificity of reactivity of the commercial antisera to free light chains was verified by staining sera from patients with known immunotypes of monoclonal gammopathies. For example, in patient sera with a distinct kappa monoclonal light chain, separate from intact monoclonal immunoglobulin, the antiserum to free kappa chains was reactive with only the kappa light chain band and did not stain the intact immunoglobulin band. The specificity was similarly confirmed for the reactivity of antiserum to free lambda light chains. The antiserum to free lambda light chains did not stain known free kappa light chain monoclonal bands and antiserum to free kappa light chains did not stain lambda monoclonal light chains.
A. MASS-FIX/MALDI test results were obtained from a reference laboratory (Mayo Laboratories, Rochester MN). The specimens provided to Mayo Laboratories were not accompanied by any clinical information.

Specimens from selected patients with high concentrations of SFLC were subjected to serial dilutions with pooled normal serum from patients younger than 30-years of age. The patients selected for serial dilutions had diagnoses of MM with detectable monoclonal light chains by conventional SIFE. One patient each with kappa and lambda light chain myeloma and one patient each with IgG kappa or IgG lambda myeloma accompanied by readily detectable free monoclonal light chains by conventional SIFE were selected. The serial dilutions applied to the sera from these patients are set forth in Tables 1-6, below.

TABLE 1
Monoclonal IgG kappa and free monoclonal kappa

| | | Mass- | Modified | Kappa | Lambda | | |
|---|---|---|---|---|---|---|---|
| | | Fix | SIFE | SFLC | SFLC | | |
| No. | Diagnosis | Result | result | Conc. | Conc. | ASCT | DARA |
| 1 | IgG K + K | IgG K | Kappa | 455.33/ | 1.59/32 | 0 | 1 |
| | (1:32)a | | monoclonal | 32 = 14.23 | | | |
| 2 | IgG K + K | IgG K | Kappa | 455.33/ | 1.59/64 | 0 | 1 |
| | (1:64)a | | monoclonal | 64 = 7.11 | | | |
| 3 | IgG K + K | IgG K | Kappa | 455.33/ | 1.59/128 | 0 | 1 |
| | (1:128)a | | monoclonal | 128 = 3.56 | | | |
| 4 | IgG K + K | Negative | Kappa | 455.33/ | 1.59/256 | 0 | 1 |
| | (1:256)a | | monoclonal | 256 = 1.78 | | | |

TABLE 2
Monoclonal IgG kappa

| | | Mass- | Modified | Kappa | Lambda | | |
|---|---|---|---|---|---|---|---|
| | | Fix | SIFE | SFLC | SFLC | | |
| No. | Diagnosis | Result | result | Conc. | Conc. | ASCT | DARA |
| 5 | IgG kappa | IgG K | Polyclonal | 32.8 | 6.67 | 0 | 0 |
| | | | kappa | | | | |
| 6 | LCP IgG K | IgG K | Kappa | 15.97 | 0.44 | 0 | 0 |
| | | Elotuzumab/LC | monoclonal | | | | |
| 7 | IgG kappa | IgG K | Kappa | 10.7 | 1.53 | 0 | 0 |
| | | | monoclonal | | | | |
| 8 | IgG kappa | IgG K | Kappa | 10 | 1.25 | 0 | 0 |
| | | | monoclonal | | | | |
| 9 | IgG kappa | IgG K | Negative | 29.48 | 0.28 | 0 | 0 |

TABLE 3
Kappa MM

| | | Mass- | Modified | Kappa | Lambda | | |
|---|---|---|---|---|---|---|---|
| | | Fix | SIFE | SFLC | SFLC | | |
| No. | Diagnosis | Result | result | Conc. | Conc. | ASCT | DARA |
| 10 | K MM | Negative | Kappa | 1224.23/ | 2.04/128 | 1 | 1 |
| | (1:128)b | | monoclonal | 128 = 9.56 | | | |
| 11 | K MM | Negative | Kappa | 1224.23/ | 2.04/256 | 1 | 1 |
| | (1:256b) | | monoclonal | 256 = 4.78 | | | |
| 12 | K MM | Negative | Negative | 1224.23/ | 2.04/412 | 1 | 1 |
| | (1:512)b | | | 512 = 2.39 | | | |
| 13 | K MM | Negative | Negative | 1224.23/ | 2.04/1024 | 1 | 1 |
| | (1:1024)b | | | 1024 1.2 | | | |
| 14 | K MM | Kappa | Kappa | 15.91 | 0.33 | 0 | 1 |
| | | | monoclonal | | | | |
| 15 | K MM | IgG | | | 0.61 | 1 | 1 |
| | | K + K − | Kappa | 47.04 | | | |
| | | Dara | mono + polyclonal | | | | |
| 16 | K MM | Negative | Polyclonal kappa | 25.42 | 0.72 | 1 | 1 |
| 17 | K MM | IgG K + K | Kappa | 16.21 | 0.59 | 0 | 0 |
| | | | monoclonal | | | | |
| 18 | K MM | IgG K + K | Kappa | 20.24 | 0.3 | 0 | 0 |
| | | | monoclonal | | | | |
| 19 | K MM | IgG K | Kappa | 17.79 | 0.2 | 0 | 0 |
| | | | monoclonal | | | | |
| 20 | K MM | Negative | Polyclonal | 11.2 | 7.23 | 1 | 0 |
| | | | K + POA mono | | | | |
| 21 | K MM | Lambda, | Polyclonal | 12.19 | 0.26 | 1 | 1 |
| | | IgG K, | K + POA mono | | | | |
| | | Dara | | | | | |
| 22 | K MM | IgG K | Negative | 2.81 | 0.37 | 0 | 0 |

TABLE 4
Monoclonal IgG lambda and free monoclonal lambda

| | | Mass- | Modified | Kappa | Lambda | | |
|---|---|---|---|---|---|---|---|
| | | Fix | SIFE | SFLC | SFLC | | |
| No. | Diagnosis | Result | result | Conc. | Conc. | ASCT | DARA |
| 23 | IgG L + L | IgG | Lambda | 2.58/32 | 293.89/ | 1 | 1 |
| | (1:32)c | L + L | monoclonal | | 32 = 9.18 | | |
| 24 | IgG L + L | IgG | Lambda | 2.58/64 | 293.89/ | 1 | 1 |
| | (1:64)c | L + L | monoclonal | | 64 = 4.59 | | |
| 25 | IgG L + L | IgG | Lambda | 2.58/128 | 293.89/ | 1 | 1 |
| | (1:128)c | L + L | monoclonal | | 128 = 2.3 | | |
| 26 | IgG L + L | IgG | Lambda | 2.58/256 | 293.89/ | 1 | 1 |
| | (1:256)c | L + L | monoclonal | | 256 = 1.15 | | |
| 27 | IgG L + L | IgG L | Lambda | 1.24 | 1.66 | 1 | 1 |
| | | | monoclonal | | | | |
| 28 | IgG L with | IgG | Negative | 0.73 | 0.92 | 0 | 0 |
| | h/o IgG | L + L | | | | | |
| | L + L | | | | | | |

TABLE 5
Monoclonal IgG or IgA lambda

| | | Mass- | Modified | Kappa | Lambda | | |
|---|---|---|---|---|---|---|---|
| | | Fix | SIFE | SFLC | SFLC | | |
| No. | Diagnosis | Result | result | Conc. | Conc. | ASCT | DARA |
| 29 | LCP IgA | IgA | Lambda | 0.41 | 19.41 | 0 | 1 |
| | Lambda | L + IgG | monoclonal | | | | |
| | | K − Dara | | | | | |
| 30 | IgG L | IgG L | Lambda | 2.08 | 5 | 0 | 0 |
| | MGUS | IgG L + | monoclonal | | | | |
| | IgG | | Lambda | | | | |
| 31 | Lambda, +Alpha | IgA L | monoclonal | 1.47 | 15.86 | 0 | 0 |

TABLE 6
Lambda MM, Biclonal Patterns, Non-monoclonal
Lambda MM

| | | Mass- | Modified | Kappa | Lambda | | |
|---|---|---|---|---|---|---|---|
| | | Fix | SIFE | SFLC | SFLC | | |
| No. | Diagnosis | Result | result | Conc. | Conc. | ASCT | DARA |
| 32 | Lambda | Lambda | Lambda | 1.28/128 | 1453.62/ | 0 | 1 |
| | MM | | monoclonal | | 128 = 11.36 | | |
| | (1:128)^{d} | | | | | | |
| 33 | Lambda | Lambda | | | | | |
| | MM | | Lambda | 1.28/256 | 1453.62/ | 0 | 1 |
| | (1:256)^{d} | | monoclonal | | 256 = 5.68 | | |
| 34 | Lambda | Lambda | Lambda | 1.28/512 | 1453.62/ | 0 | 1 |
| | MM | | monoclonal | | 512 + 2.84 | | |
| | (1:512)^{d} | | | | | | |
| 35 | Lambda | Negative | Lambda | 1.28/1024 | 1453.62/ | 0 | 1 |
| | MM | | monoclonal | | 1024 = 1.42 | | |
| | (1:1024)^{d} | | | | | | |
| 36 | L MM | Negative | Lambda | 10.63 | 14.21 | 0 | 1 |
| | | cannot | monoclonal | | | | |
| 37 | L MM | rule out | Lambda | 4.98 | 6.41 | 0 | 0 |
| | | mono | monoclonal | | | | |
| 38 | L MM | Oligo | Lambda | 11.93 | 18.28 | 0 | 0 |
| | | | mono + | | | | |
| | | | Polyclonal | | | | |
| 39 | Lambda | IgG L | Lambda | 2.1 | 11.8 | 0 | 0 |
| | restriction | | monoclonal | | | | |
| 40 | L MM | Oligo | Lambda | 8.49 | 15.17 | 1 | 0 |
| | | | monoclonal | | | | |
| 41 | L MM | IgG L | Lambda | 12.8 | 24.61 | 1 | 0 |
| | | | monoclonal | | | | |
| 42 | L MM | IgG K − | Lambda | 6.61 | 3.56 | 0 | 1 |
| | | Dara | monoclonal | | | | |

Biclonal pattern

| | | | | | | | |
|---|---|---|---|---|---|---|---|
| 43 | IgG K + | IgG K + | Kappa | 12.53 | 4.32 | 0 | 0 |
| | IgG L | IgG L | monoclonal | | | | |

Non monoclonal

| | | | | | | | |
|---|---|---|---|---|---|---|---|
| 44 | Polyclonal | Negative | Negative | 21.71 | 11.19 | NA | NA |
| | hyper | | | | | | |
| | gamma | | | | | | |
| 45 | Polyclonal; | Negative | Negative | 55.9 | 19.62 | NA | NA |
| | ESRD | | | | | | |
| 46 | Polyclonal- | Negative | Negative | 11.86 | 12.15 | NA | NA |
| | Sjogren | | | | | | |
| 47 | Pooled | Negative | Negative | 8.52 | 6.63 | NA | NA |
| | normal | | | | | | |
| | serum | | | | | | |

For Tables 1-6: The column labeled “Diagnosis” provides the primary diagnosis of the monoclonal gammopathy. The superscripts a-d denote various dilutions from a single patient in each group. The dilution is indicated in parenthesis, and calculated concentration of involved SFLC is listed. Lambda restriction in this column refers to a patient with a lambda monoclonal band on SIFE without a cognate heavy chain. The laboratory and clinical findings did not support a diagnosis of lambda MM.

The next two columns list the results from MASS-FIX/MALDI as provided by Mayo Laboratories and those obtained by FLC-Modified SIFE.

The columns Kappa and Lambda SFLC denote the results from SFLC assay. For specimens that were serially diluted, the raw SFLC concentration, dilution factor, and the estimated concentration of the involved monoclonal light chains are given, in order. The estimated concentration of monoclonal light chain is noted for only the involved light chain.

L=lambda light chain; K=kappa light chain.

ASCT— Autologous stem cell transplantation. 1=ASCT done, 0=ASCT not done.

DARA denotes patient treated with daratumumab. 1=Dara administered; 0=Dara not administered. No other therapeutic monoclonal antibody was used for the treatment of patients reported here.

POA refers to point of application. If a light chain monoclonal band was noted at the point of application, this observation was noted as a caution to not over-interpret an artefact at the point of application as a monoclonal band.

NA=Not applicable.

A summary of parallel testing results is given in Table 7, below.

TABLE 7
Summary of results of comparison of MASS-FIX/MALDI and FLC-
Modified SIFE for detection of monoclonal light chains.

| | |
|---|---|
| No. of specimens with monoclonal Ig | 43 |
| Positive for monoclonal LC by both methods | 18 |
| Positive with FLC-Modified SIFE only | 24 |
| Positive with MASS-FIX/MALDI only | 1 |
| No. of specimens without monoclonal Ig | 4 |
| Negative by both methods | 4 |

Results

FLC-Modified SIFE revealed monoclonal light chains in consonance with the expected findings, given a patient's diagnosis and immunoglobulin type determined by conventional SPEP and SIFE. Representative results from patients with light chain myeloma and intact immunoglobulin lesions with a separate band representing free monoclonal light chains noted by SIFE are shown in FIGS. 1A-1J and FIGS. 2A-2B. FIG. 1A exhibits the typical staining pattern of monoclonal free light chains in serum. FIG. 1B demonstrates the staining pattern of polyclonal light chain (Upper fainter, diffuse stained area) and a monoclonal free light chain (Lower, sharper, darker staining band). FIGS. 1C-1F shows serial dilutions of serum from a patient with IgG Lambda MM and free monoclonal lambda light chains show the progressive loss of intensity of staining. Serial dilutions allowed to determine lower limit of detection for lambda light chains, by this method. FIGS. 1G-1J shows Serial dilutions of serum from a patient with Kappa MM show the progressive loss of intensity of staining. Serial dilutions allowed to determine lower limit of detection for kappa light chains, by this method.

FIGS. 2A and 2B show Representative gels from FLC-Modified SIFE are presented. The lanes marked SP, G. A, M, κ, λ, represent conventional serum immunofixation gels stained with appropriate antisera. The lanes marked anti-free k and anti-free λ represent FLC-Modified SIFE, stained with antisera to respective free light chains. In FLC-Modified SIFE, undiluted patient serum was applied and following staining with respective antisera, the gels were washed three times. The lane marked anti-free k shows free monoclonal light chains in the same location as the intact immunoglobulin (FIG. 2A). Please note that in the lane stained with anti-free kappa, undiluted serum was applied, whereas in conventional SIFE G and K lanes were at 1:10 dilution of serum. The broader band in the anti-free kappa lane represents the higher dose of inoculum. In FIG. 2B, conventional SIFE shows monoclonal IgG λ and free monoclonal λ light chains in a separate anodal band. Only the monoclonal λ light chain band was stained with the antiserum to free λ light chains.

Serial dilutions of serum from patient “a”, in Tables 1-6, revealed the limit of detection of kappa monoclonal light chains to be about 1.78 mg/L. This patient's primary diagnosis was IgG kappa MM and conventional SIFE revealed a separate band of monoclonal kappa light chains. MASS-FIX/MALDI detected monoclonal IgG kappa but did not identify free monoclonal kappa light chains. In patients with documented IgG kappa MM and kappa light chain MM, FLC-Modified SIFE displayed more results in consonance with the primary lesions. Serial dilutions of kappa LCMM, in patient “b”, identified a monoclonal band by FLC-Modified SIFE at a total SFLC concentration of 4.78 mg/L. MASS-FIX/MALDI did not detect monoclonal light chains at SFLC concentration of 9.56 mg/L, the highest concentration tested in this patient. On the other hand in some patients with kappa and lambda LCMM, MASS-FIX/MALDI identified intact monoclonal IgG kappa, or IgG lambda, when none was expected, based on the sum total of laboratory and clinical findings, e.g., specimen #s 15, 17-19, 21, 22, 39, 41, 42. In a one case each, monoclonal lambda was noted by MASS-FIX/MALDI in kappa MM and IgG kappa in lambda MM, specimen #21, 42 (Tables 1-6).

Serial dilutions of serum from patient “c” revealed monoclonal lambda light chain by both FLC-Modified SIFE and MASS-FIX/MALDI at a concentration of about 1.15 mg/L. MASS-FIX/MALDI identified free lambda light chains in a patient with history of monoclonal IgG lambda and free monoclonal lambda light chains when FLC-Modified SIFE did not detect monoclonal lambda light chains. The total SFLC concentration of free lambda light chain was 0.92 mg/L in the specimen addressed above. Serial dilutions of serum from patient “d” with a diagnosis of lambda LCMM revealed monoclonal lambda light chains to a concentration of 1.42 mg/L by FLC-Modified SIFE while MASS-FIX/MALDI was positive to a concentration of 2.84 but not at 1.42 mg/L.

In a patient with a biclonal pattern of IgG kappa and IgG lambda, FLC-Modified SIFE detected monoclonal kappa light chains. Staining with anti-lambda antiserum did not detect free monoclonal lambda light chains. MASS-FIX/MALDI identified both the intact monoclonal immunoglobulins, i.e., IgG kappa and IgG lambda, but did not detect free monoclonal light chains of either type, specimen #43.

As expected, in three patients with polyclonal increase in immunoglobulins and a specimen from pooled sera did not reveal a monoclonal light chain by either method.

The summary of comparative findings by FLC-Modified SIFE and MASS-FIX/MALDI are shown in Table 7 and demonstrate superior detection of monoclonal light chains by the FLC-Modified SIFE method.

Summary of results of all specimens tested by FLC-Modified SIFE are shown in Table 8. Briefly, monoclonal kappa light chains were detected in specimens with total SFLC concentration as low as 1.78 mg/L for kappa and 1.15 mg/L for lambda. The highest levels of SFLC in patients with monoclonal gammopathy and negative results by FLC-Modified SIFE were 29.48 mg/L for kappa and 10.51 mg/L for lambda light chains. The highest levels of SFLC observed in patients, without detection of monoclonal light chains, were commonly seen in patients with end stage renal disease (ESRD). There was no evidence of monoclonal gammopathy in these patients. A similar phenomenon was also noted in patients with other conditions associated with polyclonal increase in gamma globulins, e.g., rheumatologic disorders and cirrhosis. The highest levels associated with negative results for monoclonal light chains, in patients without a diagnosis of monoclonal gammopathy, by FLC-Modified SIFE assay were 128.18 mg/L for kappa and 57.76 mg/L for lambda light chains.

TABLE 8
Summary of results of all specimens tested by FLC-Modified SIFE

| LC type, in NMG | N | Lowest Conc. Pos. | Highest Conc. Neg. |
|---|---|---|---|
| Kappa | 87 | 1.78 | 29.48 |
| Lambda | 56 | 1.15 | 10.51 |
| Polyclonal | 19 | NA | K = 128.18; L = 57.76 |

Progress in treatment of MM and the potential for a curative treatment with CAR-T therapy warrants improvement in methods for detecting minimal residual disease. Mass Spectrometric analysis following nanobody mediated concentration of immunoglobulins (MASS-FIX/MALDI) has been described as a method for improved sensitivity and detection of MRD, though the results of MASS-FIX/MALDI were not compared with a reference method or gas chromatography mass-spectrometry, or even urine examination. This method has also been promoted for use as a screening method for monoclonal gammopathies in lieu of SPEP and SIFE; and UPEP and UIFE, the current “gold standard”/reference method.

A method using ultrafiltration to separate and concentrate SFLC followed by SIFE and densitometric scanning (QUIET) has been shown to have a detection limit of about 1.0 mg/L of monoclonal SFLCs (Singh, et al., Lab. Med. 2020; 51:592-600). Prompted by this prior investigation, the use of polyclonal antiserum to free light chains was explored for detecting free monoclonal light chains. To improve the sensitivity of the method, undiluted patient serum was applied to SIFE gels. It is noted that the manufacturer's protocol for SIFE entails 1:10 dilution of patient serum for staining with antisera to gamma heavy chains and kappa light chains and 1:5 dilutions for staining for mu, and alpha heavy chains and lambda light chains. The use of undiluted patient serum required additional wash steps for removing excess proteins not reacting with the antiserum. In preliminary studies it was noted that high concentrations of intact monoclonal immunoglobulins sometimes produced false positive result due to the monoclonal immunoglobulin not being washed out by the conventional SIFE protocol. The FLC-Modified SIFE procedure described here obviates such false positive results. In some patients with negative results on staining with antisera to free light chains, a triple application of the patient serum to SIFE gel was carried out to confirm the negative results.

While the specificity of reactivity of antisera to free light chains to the respective light chain was tested, the possibility of a rare cross reactivity with other light chain could not be excluded, i.e. antiserum to free kappa light chain reacting with free lambda light chains. A more likely outcome is of a false negative result due to lack of reactivity of antiserum to free light chains of a given clonotype. This outcome was observed in one patient whose monoclonal light chain did not react with antiserum from one manufacturer, but did react with antiserum from a different manufacturer. The reverse was also observed in a different case.

In patients with lambda light chain associated lesions, either lambda chain MM or IgG lambda or IgA lambda MM, kappa free light chain concentration was sometimes higher than lambda SFLC concentration following treatment, especially ASCT. In one such patients with kappa dominant kappa/lambda ratio, for example, sample #42, detection of monoclonal lambda light chain and lack of detection of monoclonal kappa chain by FLC-Modified SIFE is likely to be due to the high levels of kappa light chains being all polyclonal while all, or most, of the lambda light chains being monoclonal. The post-ASCT treatment abundance of polyclonal kappa light chains, including in patients with lambda light chain associated lesions, has been documented earlier. (Lee and Singh J Clin Med Res. 2018; 10:562-569; Lee and Singh, Lab Med. 2019; 50:381-389 DOI: 10.1093/labmed/lmz007).

The lack of detection of monoclonal light chains, by FLC-Modified SIFE, in patients with monoclonal gammopathy and SFLC concentrations above the detection limit of the method are postulated to be due to lack of sufficient amount of monoclonal light chains and the bulk of SFLC being polyclonal in nature, as is often observed in sera following treatment. For instance, in this context, it is likely that the bulk of kappa SFLC are polyclonal in sample #9; thus, this would explain the lack of detection of monoclonal kappa light chains in this patient. Both polyclonal and monoclonal light chains were detected in some of the patients, as depicted in Tables 1-6 and illustrated in FIGS. 1A-1J.

To assess the relative sensitivity of the FLC-Modified SIFE procedure, samples were tested in parallel by MASS-FIX/MALDI at a reference laboratory (Mayo Laboratories, Rochester, MN). In this parallel evaluation de-identified specimens without clinical data were provided to the reference laboratory. As indicated in the results in Tables 1-6, there was only one specimen in which MASS-FIX/MALDI detected a free monoclonal lambda light chain that was not detected by FLC-Modified SIFE. This patient had IgG lambda MM and had a history of monoclonal IgG lambda and free monoclonal lambda lights, earlier in the course of disease. In the sample noted as specimen #28, conventional SIFE was negative, and FLC-Modified SIFE did not detect monoclonal lambda light chains. Total lambda SFLC concentration in this patient was 0.92 mg/L and was apparently below the detection limit of FLC-Modified SIFE. In all other cases of disparate results between FLC-Modified SIFE and MASS-FIX/MALDI, the results with FLC-Modified SIFE displayed greater sensitivity for detection of monoclonal free light chains. In these instances, the higher sensitivity for detection of monoclonal free light chains by FLC-Modified SIFE is bolstered by the results on serially diluted patient sera as documented in Tables 1-6. Higher sensitivity by the FLC-Modified SIFE was always in consonance with the clinical findings and expected/predicted results. The FLC-Modified SIFE was not designed to test for intact monoclonal immunoglobulins and it was not possible to assess the results of MASS-FIX/MALDI showing monoclonal intact immunoglobulin when none was expected, as was seen in patients with light chain MM. However, detection of monoclonal intact immunoglobulins by MASS-FIX/MALDI in patients with LCMM raises the question of the validity of the method in detecting MRD. It is possible that intact monoclonal immunoglobulins detected in LCMM patients represent oligoclonal pattern in patients status-post chemotherapy and/or ASCT. To use the presence of monoclonal intact immunoglobulin in patients with MM would warrant proof that the monoclonal Ig detected by MASS-FIX/MALDI is identical to the original malignant clone. At this stage reporting of intact monoclonal immunoglobulins in patients with light chain myeloma could be considered a false positive result, casting doubt on the validity of MASS-FIX/MALDI detected monoclonal intact immunoglobulins indicating residual disease. This would be especially applicable to patients who had not undergone ASCT.

The lower sensitivity of MASS-FIX/MALDI is likely to be a function of the limited repertoire of antibody activity in the camelid antisera/nanobodies used to enrich the pool of immunoglobulins. The nanobodies may not recognize certain unique epitopes in some monoclonal free light chains. This hypothesis is supported by findings in a limited number of patient specimens tested in which the expected monoclonal free light chain was not detected by one reagent antiserum but was detectable by a second antiserum from a different vendor. It is likely that improved nanobodies with a broader repertoire of reactivity towards the unique epitopes in monoclonal light chains may improve the sensitivity of MASS-FIX/MALDI for expanded detection of free monoclonal light chains.

With respect to diagnostic algorithms for monoclonal gammopathy, it is conceivable that using usual SPEP, SIFE and FLC-Modified SIFE could detect all specimens with pathologic monoclonal immunoglobulins, including light chain only lesions. To maximize resource utilization, initial screening studies could be performed with SPEP and a single lane of undiluted patient serum stained with a mixture of anti-kappa and anti-lambda antisera. Serum specimens with a positive result by SPEP or FLC-Modified SIFE using mixtures of anti-kappa and anti-lambda antisera could be further analyzed by conventional SIFE, UPEP and UIFE, and FLC-Modified SIFE with two lanes, one each for antibodies to kappa and lambda free light chains.

While the data indicate that the FLC-Modified SIFE method shows promise in improving detection of free monoclonal light chains, there are several potential caveats. Among the potential concerns regarding the use of the FLC-Modified SIFE assay are (a) increased labor to carry out multiple washes in addition to those in the conventional SIFE procedure, (b) cost of antisera to free light chains, and (c) modification in the reporting process for the electrophoresis algorithm. In this context, it may be appropriate to invoke this method only if and when detection of minimal residual disease becomes a clinically relevant issue. FLC-Modified SIFE would be used only if conventional SIFE and UIFE were negative for monoclonal light chains, or if urine was not available. The increase in technologist time amounts to about one additional hour per gel for the FLC-Modified SIFE procedure. Such an increase could be accommodated at most academic medical centers without the need to hire additional personnel. The increase in cost of reagents would be about $11.25 for one specimen and one antiserum type. This would be lower than the current cost of commercial MASS-FIX/MALDI at $145.00/specimen for a test with lower sensitivity. In-house testing would allow the cost to be recovered, once the test is approved by Centers for Medicare and Medicaid, through billing.

The salient findings in comparison with MASS-FIX/MALDI document frequent lack of detection of monoclonal free light chains by MASS-FIX/MALDI. In addition, MASS-FIX/MALDI detected intact monoclonal immunoglobulins in some patients with light chain only myeloma, even when the patient had not undergone ASCT. More concerning was the detection of monoclonal lambda light chains in specimen from a patient with Kappa MM. (Specimen #21). Monoclonal IgG kappa, in addition to DARA, was reported in a specimen from a patient with lambda MM who had not received ASCT, specimen #42. The ASCT treatment can induce oligoclonal pattern that could be interpreted as monoclonal intact immunoglobulin. Also, of concern was detection of Elotuzumab in specimen #6 when the patient had not received this therapeutic monoclonal antibody.

### Example 2: Free Light Chain Urine Immunofixation Electrophoresis (FLC-UIFE) is More Sensitive than Conventional UIFE Assay for Detecting Monoclonal Light Chains

Monoclonal immunoglobulin light chains in serum and urine are a marker for monoclonal gammopathy and could serve as markers of residual/minimal residual disease in multiple myeloma. Excretion of monoclonal light chains in urine is known to result in renal damage and shorter survival in patient with light chain predominant multiple myelomas. Retrospective review of urine immunofixation in 1522 specimens, at three medical centers was conducted to assess the utility of urine examination for diagnosis of monoclonal gammopathy. 228, stored, urine specimens were tested by modified urine immunofixation method (FLC-UIFE) using antisera specific to free light chains.

Methods

The study was conducted at 3 medical school-affiliated hospitals in the southeastern (institution A), midwestern (institution B), and northern (institution C) United States. The investigation consisted of retrospective review of UIFE and SPEP+SIFE results to ascertain the value added by UIFE examination. Total urine protein (TPr) and SFLC levels were also documented and analyzed for diagnostic utility. Stored, concentrated urine specimens, previously tested by routine UIFE, were examined via the FLC-UIFE method, as described later in this article, at institution A. The SPIFE Touch System (Helena Laboratories) was used for SPEP, SIFE, UPEP, and UIFE at institution A. Institutions B and C used the CAPILLARYS system (Sebia) for SPEP and IFE. SFLC quantification was conducted by using binding-site reagents and Optilite (The Binding Site Group). The study protocol was reviewed by relevant institutional review boards, which considered the proposal to be exempt.

Data from the 3 institutions were analyzed separately. The involved LC identity of the relevant monoclonal immunoglobulin was ascertained from the results of SPEP/SIFE testing and designated as LC in the data tables. The levels of SFLC at the time of UIFE were recorded as mg/L. TPr concentration, before further concentration, in the specimens analyzed was recorded as mg/dL.

The UIFE results were scrutinized for additional information gleaned from UIFE that was not available from SPEP/SIFE and UPEP examination. In particular, the presence of a distinct band of monoclonal free light chains (MFLCs) was noted as such. In specimens in which MFLC bands were detectable, comparison with specimens without such bands for the concentration of TPr, cognate SFLC concentration, and ratio of involved to uninvolved SFLC concentration, the lowest level of cognate SFLC, lowest ratio of involved to uninvolved SFLC, and lowest TPr concentration associated with the presence of MFLC in urine by conventional UIFE were recorded. Similarly, the highest level of cognate SFLC, the highest ratio of involved to uninvolved SFLC, and the highest concentration of urine protein associated with lack of MFLC in UIFE were determined. Thus, the investigation included of retrospective review of
(a) UIFE and
(b) SPEP+SIFE results to ascertain the value added by UIFE examination.
(c) Total urine protein and
(d) SFLC levels were also documented and analyzed for diagnostic utility.
(e) Stored, concentrated urine specimens, previously tested by routine UIFE, were examined by FLC-UIFE method, as described below, at only one of the medical centers.

Free Light Chain Urine Immunofixation Electrophoresis (FLC-UIFE)

For FLC-UIFE, urine specimens submitted for routine patient testing were concentrated by membrane filtration with Millipore Concentrators (Merck) and tested by conventional UPEP and UIFE. Residual specimens were stored at 4° C., and selected specimens were evaluated via FLC-UIFE. Only specimens from patients with monoclonal gammopathy or a history of monoclonal gammopathy were selected. Concentrated urine was applied to UIFE gels procured from Helena Laboratories, and the SPIFE Touch instrument was used to electrophorese the specimens, as is usually performed in conventional UIFE. Instead of antisera provided by the manufacturer with the UIFE/SIFE kits, the gels were stained with antisera to SFLCs. The anti-kappa and anti-lambda antisera to FLCs were obtained from Sebia. 50 μL of undiluted antisera was applied in the slots in the UIFE/SIFE template. Other than using antisera specific to FLCs, the FLC-UIFE protocol was similar to conventional UIFE. Incubation with antibody and staining were conducted per the standard protocol for UIFE using the Helena platform. The stained gels were evaluated visually and the results compared with those yielded by previously conducted conventional UIFE.

Residual specimens were stored at 4° C. and selected specimens were evaluated by FLC-UIFE. Only specimens from patients with a monoclonal gammopathy or history of monoclonal gammopathy were selected and processed as follows:
(a) Concentrated urine was applied to UIFE gels procured from Helena Laboratories (Beaumont TX),
(b) SPIFE Touch, was used to electrophorese the specimens as usually done for conventional UIFE.
(c) Instead of antisera provided by Helena, with the UIFE/SIFE kits, the gels were stained with antisera to serum free light chains. The anti-kappa and anti-lambda antisera to free light chains were procured from Sebia Inc. (Norcross GA). 50 μL of undiluted antisera were applied in the slots in the UIFE/S IFE template.
(d) Incubation with antibody and staining were conducted as is usually done for UIFE in the Helena equipment.
(e) The stained gels were evaluated visually and the result compared with previously conducted conventional UIFE.

Data from each of the institutions were analyzed separately. The involved light chain identity of the relevant monoclonal immunoglobulin was ascertained from the results of SPEP/SIFE and designated as LC in the data tables. The levels of SFLC at the time of UIFE were recorded as mg/L. Total urine protein concentration, prior to concentration, in the specimens analyzed was recorded as mg/dL. The UIFE results were scrutinized for additional information gleaned from UIFE that was not available from SPEP/SIFE examination.

The presence of a distinct band of monoclonal free light chains was noted as monoclonal free light chains (FLC). In specimens in which monoclonal free light chain bands were detectable were compared with specimens without such bands for the concentration of total urine protein, cognate SFLC concentration, and ratio of involved to uninvolved SFLC concentration. The lowest level of cognate SFLC, lowest ratio of involved to uninvolved SFLC and lowest total urine protein concentration associated with the presence of monoclonal free light chains in urine by conventional UIFE were recorded. Similarly, the highest level of cognate SFLC, highest ratio of involved to uninvolved SFLC, and highest concentration of urine protein associated with lack of monoclonal free light chains in UIFE were ascertained.

The results of conventional UIFE were also compared to the results from FLC-UIFE at one of the institutions. Detection of monoclonal free light chains by the two methods were compared and contrasted. Urine specimens collected from January 2020 through March 2022 at institution “A”, for which sufficient volume for testing was available, were evaluated for testing by FLC-UIFE. Patients with extant monoclonal immunoglobulins or a history of positive SPEP/SIFE or UIFE for monoclonal Ig were included. A total of 228 specimens, all from institution “A”, were tested by FLC-UIFE and results compared with recorded results of convention UIFE. Rate of additional positive results between kappa and lambda light chains was analyzed by Chi Squared (X²) Test.

Results

The details of this study are described in “Urine Protein Immunofixation Electrophoresis: Free Light Chain Urine Immunofixation Electrophoresis Is More Sensitive than Conventional Assays for Detecting Monoclonal Light Chains and Could Serve as a Marker of Minimal Residual Disease”, by Singh et al., Laboratory Medicine, 2023; lmac155, the contents of which are specifically incorporated herein in their entirety.

In the review of 1738 UIFE examinations, the only meaningful additional information in UIFE was the presence of MFLCs in a variable proportion of specimens. The proportion of specimens with MFLCs was markedly different among the 3 institutions. At institution A, only 17.6% of the specimens displayed a band of MFLCs; at institution B, the corresponding figure was 88.7%; and at institution C, the corresponding value was 39.8%. The distribution of diagnoses at institution A is given in Table 9.

The vast majority of the patients had multiple diagnoses. A prototypic pattern was for the patient to have hypertension, diabetes, and chronic kidney disease, as well as having a body mass index in the overweight category. Patients with multiple pathologies are listed in the multimorbid category. The diagnosis listed in the table represents the likely diagnosis prompting the UIFE study. Patients with monoclonal gammopathy disorders were often tested multiple times, whereas other patients were usually tested only once. Examination of 228 urine specimens by the new method revealed 18% additional positive findings that were in consonance with the clinical data. The rate of additional findings for lambda light chains was nearly three times higher than for kappa light chains.

TABLE 9
Clinical Diagnoses, Affected Organ Systems, and
Distribution of Diagnoses in the Patient Population
at the Main Study Site (Institution A)

| Clinical Diagnosis/Diagnoses | % |
|---|---|
| Neurologic disorders | 22.7 |
| MM | 13.6 |
| Chronic kidney disease | 12.5 |
| Hematological disorders without monoclonal gammopathy | 9.2 |
| Multimorbid/polymorbid | 7.0 |
| Rheumatologic disorder | 5.6 |
| Cardiac disorders | 5.5 |
| MGUS | 4.7 |
| Osteoporosis | 4.5 |
| Infections, including HIV and sepsis | 3.6 |
| Amyloidosis | 3.2 |
| Gastroenterology lesions | 3.1 |
| Endocrine disorders | 3.0 |
| Bone lesions | 2.7 |
| Other renal disorders | 2.5 |
| Psychiatric disorders | 1.1 |
| Smoldering MM | 0.7 |
| Skin disorders | 0.7 |
| Gynecologic disorders | 0.6 |
| Urologic disorders | 0.4 |
| ENT lesions | 0.2 |
| Respiratory disorders | 0.2 |

ENT = ear, nose, and throat; MGUS = monoclonal gammopathy of undetermined significance; MM = multiple myeloma.

A summary of the findings with respect to relation between the presence and absence of monoclonal free light chains in urine at the different intuitions to other parameters is shown in Table 10. The lowest concentration of the cognate LC with a positive result for MFLCs was 0.56 mg/L for kappa and 1.03 mg/L for lambda. The highest concentration of cognate LC with a negative UIFE result was 502.71 mg/L for kappa and 94.39 mg/L for lambda. The lowest ratio of concentration of involved and uninvolved (I/U) SFLC in urine testing positive for FMLCs via conventional UIFE was 0.0018 for kappa and 0.0073 for lambda. The highest ratio of concentration of I/U SFLC in patients with urine that tested negative for MFLCs by conventional UIFE was 609.56 for kappa and 222.68 for lambda. The lowest concentration of TPr in a specimen containing MFLCs of either type was 2.0 mg/dL. The highest concentration of TPr in a specimen testing negative for MFLCs of either type was 7428.0 mg/dL.

The proportion of specimens testing positive for MLCs, via the FLC UIFE method, at total protein concentration of <5.0 mg/dL, <10.0 mg/dL, <15.0 mg/dL, <20.0 mg/dL, and <30.0 mg/dL are shown in Table 11. The proportion of urine specimens with detectable MFLCs detected via conventional UIFE is also noted in Table 10, and it varies markedly among institutions.

TABLE 10
Urine and SFLC Parameters Regarding Presence or Absence
of Detectable MLCs in Urine via Conventional UIFE^{a}

| | Institution | Institution | Institution |
|---|---|---|---|
| Conventional UIFE Results | A | B | C |
| | LC Conc, mg/L | | |
| Lowest conc at which kappa | 1.19 | 0.56 | 0.57 |
| positive | | | |
| Lowest conc at which lambda | 1.03 | 1.23 | 1.44 |
| positive | | | |
| Highest conc at which kappa | 502.71 | 213.27 | 54.12 |
| negative^{b} | | | |
| Highest conc at which lambda | 75.4 | 94.39 | 15.73 |
| negative^{b} | | | |
| Urine protein lowest conc | 4 | 5 | 2 |
| positive | | | |
| Urine protein highest conc | 7428^{c} | 352 | 524.4 |
| negative | | | |
| I/U Ratio | | | |
| Lowest K/L conc at which kappa | 0.059 | 0.002 | 0.129 |
| positive | | | |
| Lowest L/K conc at which lambda | 0.007 | 0.114 | 0.806 |
| positive | | | |
| Highest K/L conc at which kappa | 274.7 | 609.56 | 82 |
| negative | | | |
| Highest L/K conc at which lambda | 119.68 | 222.68 | 62.5 |
| negative | | | |
| | No. (%) | | |
| No monoclonal LCs detected in | 1128 (82.0) | 17 (11.3) | 86 (39.8) |
| urine | | | |
| Kappa monoclonal LCs | 143 (10.4) | 87 (58.0) | 88 (40.7) |
| Lambda monoclonal LCs | 99 (7.2) | 44 (29.3) | 40 (18.5) |
| Undetermined monoclonal LCs, | 2 | 2 | 0 |
| No. | | | |
| Total No. of specimens | 1372 | 150 | 216 |

Conc = concentration; I/U = involved/uninvolved; LC = light chain; MLC = monoclonal light chain; SFLC = serum free light chain; TPr = total urine protein; UIFE = urine immunofixation electrophoresis.
^{a}SFLC is expressed as mg/L, and TPr is measured as mg/dL.
^{b}The high SFLC values with negative results via conventional UIFE are due to comigration of the relevant MLC with the intact immunoglobulin and do not imply the absence of MLCs. FLC-UIFE would identify MLCs in such circumstances.
^{c}This unusually high TPr value was from a patient with nephrotic syndrome, when seen in the emergency department. Follow-up specimens were not available. The next 2 highest urine protein values with negative UIFE results, from different patients, were 2965 mg/L and 2226 mg/dL.

TABLE 11
TPr Values for Specimens Testing Positive for MLCsa,

| Total Positive at TPr Value | No. (%) | |
|---|---|---|
| ≤5 mg/dL | 13 | (5.1) |
| ≤15 mg/dL | 47 | (18.5) |
| ≤20 mg/dL | 58 | (22.8) |
| ≤30 mg/dL | 79 | (31.1) |
| Total | 254 | (100) |

MLCs = monoclonal light chains; TPr = total urine protein; UIFE = urine immunofixation electrophoresis.
^{a}Including the specimen that tested positive via UIFE only.
^{b}Approximately one-third of the specimens displayed MLCs despite having urine protein values in the normal range.

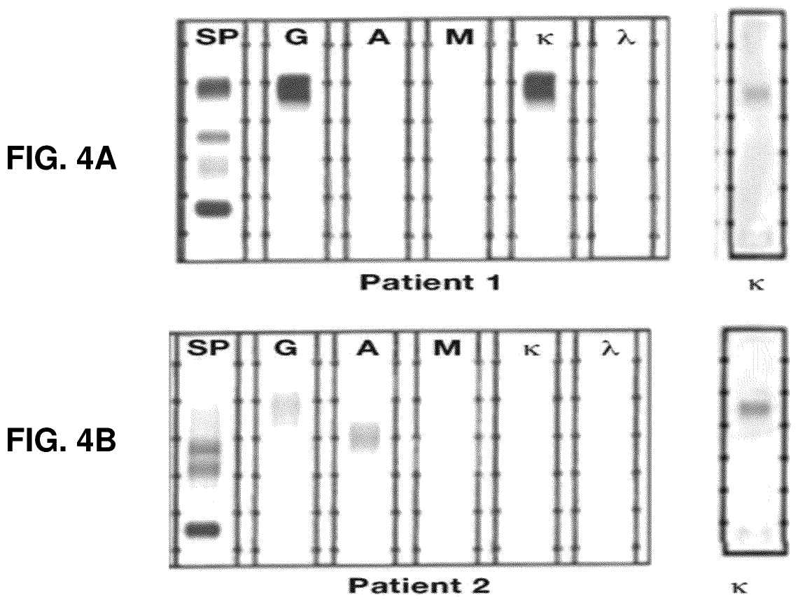

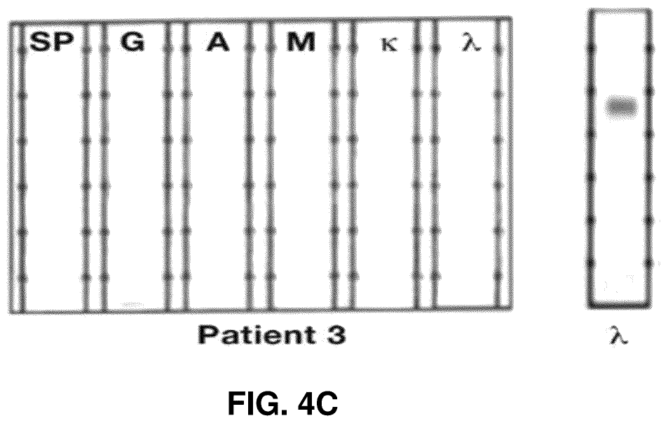

Comparison of conventional UIFE with FLC-UIFE done with antisera for free kappa and lambda light chains is shown in Table 12. In a single instance of a patient with IgM Kappa monoclonal immunoglobulin, the conventional UIFE was positive for monoclonal kappa light chains but FLC-UIFE was negative. The specimen was retested by conventional UIFE to exclude deterioration of the specimen being responsible for negative result on FLC-UIFE. Repeat testing confirmed the presence of monoclonal kappa light chains by conventional UIFE and a negative result by FLC-UIFE. This was observed despite the fact that FLC-UIFE had a higher sensitivity as demonstrated by a positive result in a patient with SFLC level of 0.48 mg/dL whereas the lowest SFLC concentration with a positive result on conventional UIFE for monoclonal free kappa light chains had a SFLC level of 1.19 mg/dL. (FIGS. 3 and 4A-4C). FIG. 3 shows results of analyzing urine from a patient with IgM Kappa monoclonal immunoglobulin in serum, tested by conventional UIFE and staining with anti-kappa antibody (presented in lane A) showing a low intensity monoclonal kappa light chain. No kappa light chain was detected with antiserum to free kappa light chains (presented in lane B). FIGS. 4A-4C show results of conventional UIFE and FLC-UIFE from three patients. The lanes marked SP, G, A, M, K, and L represent conventional UIFE. The unmarked separate lane in first two patients were stained with anti-serum to free kappa light chains and in the third patient for free lambda light chains. The lack of detection of free monoclonal kappa light chain in patient 1 by conventional UIFE is due to overlap in the location of intact monoclonal IgG K and K free monoclonal light chain band. The detection of monoclonal kappa and lambda light chain bands in patients 2 and 3 reflects the greater sensitivity of FLC-UIFE over conventional UIFE.

TABLE 12
Comparison of performance or conventional UIFE and FLC-
UIFE in detecting monoclonal light chains in urine.

| Conventional UIFE vs FLC-UIFE Testing for MLCs | Kappa | Lambda |
|---|---|---|
| Specimens with LC type per SPEP/SIFE, No. | 140 | 88 |
| Specimens testing positive via conventional UIFE, No. | 52 | 24 |
| Specimens testing positive via FLC-UIFE, No. | 67 | 51 |
| Lowest LC conc with positive results, mg/L | 0.48 | 1.03 |
| Highest LC conc with negative results, mg/L | 38.27 | 37.92 |
| Lowest I/U ratio with positive results | 0.82 | 0.396 |
| Highest I/U ratio with negative results | 105.29 | 35 |
| Urine protein lowest conc with positive results | 4 mg/dL | |
| Urine protein highest conc with negative results | 7428 mg/dL | |
| Additional cases of MLC identified via FLC-UIFE, | 15 | 27 |
| No. | | |
| Additional FLC-UIFE positive results, %^{a} | 10.7% | 30.7% |

| | |
|---|---|
| Total additional FLC-UIFE positive results, No. (%) | 42 (18.4%) |

FLC = free light chain; I/U = involved/uninvolved; LC = light chain; MLCs = monoclonal light chains; SIFE = serum immunofixation electrophoresis; SPEP = serum protein electrophoresis; UIFE = urine immunofixation electrophoresis.
^{a}The greater number of lambda-positive specimens, compared with kappa-positive specimens, was statistically significant, at P < .001. The greater rate of positivity for lambda MLCs was predominantly due to greater sensitivity of antiserum to lambda FLCs than the antilambda antiserum in Helena Laboratories kits, and only partly due to comigration of lambda MLCs with intact monoclonal immunoglobulin.
^{b}P < .001.

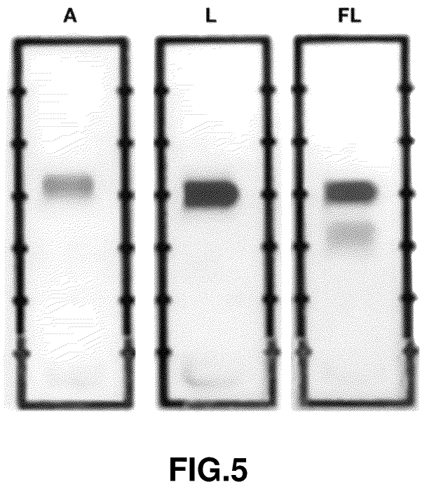

FLC-UIFE detected MLCs in approximately 18% more specimens vs conventional UIFE. The higher positive rate is statistically significant, at P<0.001. As noted earlier herein, there was only 1 instance in which FLC-UIFE failed to detect kappa MLCs in urine, and conventional UIFE detected them. Also, lambda MLCs were detected by FLC-UIFE at almost 3 times the rate at which additional kappa MLCs were detected-29% vs 11%. The higher rate of detection for lambda MLCs, compared with kappa LCs, was significant, at P<0.001. The higher rate of detection of MFLCs is partly due to comigration of MLCs and corresponding monoclonal intact immunoglobulin and the MLC band being obscured by intact monoclonal immunoglobulin (FIGS. 2 and 3). FLC-UIFE also detected MFLCs not stained by conventional UIFE reagents (FIGS. 4A-4C and FIG. 5).

As depicted in FIG. 5, when conventional serum immunofixation electrophoresis (SIFE) and free light chain (FLC)—SIFE from the patient with the highest level of lambda FLCs at institution A were compared, conventional UIFE was interpreted to contain monoclonal IgA lambda; lambda monoclonal free light chains (MFLCs) comigrated with the intact monoclonal IgA lambda. As depicted by the different degrees of staining between lanes A, L and FL in FIG. 5, staining in the L lane is darker, however that fact by itself was considered insufficient to conclude the presence of lambda MFLCs. The upper, darker band in lane FL represents lambda MFLCs migrating at the same location as intact IgA lambda. The lower, fainter band in the FL lane represents separate, lower concentration of lambda MLCs with different mobility, likely due to alterations in serum or urine. The altered lambda MFLCs did not react with the antilambda antiserum in the Helena IFE kits. This case illustrates the 2 mechanisms of higher sensitivity of FLC-UIFE: monoclonal light chains (MLCs) comigrating with intact monoclonal Ig, which can be identified as MFLCs due the specificity of the antiserum for free light chains (FLCs); altered MFLCs in urine were detectable with antiserum to FLCs but not via the conventional reagents. These 2 mechanisms were variably applicable in cases in which FMLCs were detected by Sebia antisera to FLCs but were not detectable by conventional antisera in Helena IFE kits.

The relationship of the kappa/lambda LC ratio recommended by The Binding Site, with respect to the presence and absence of detectable MLCs with FLC-UIFE, is shown in Table 13. A normal ratio does not exclude MLCs, and an abnormal ratio is not diagnostic of monoclonal gammopathy yielding high false-negative and high false-positive rates.

TABLE 13
Performance of FLC-UIFE with Respect to K/L Ratio

| Result | No. (%) |
|---|---|
| Kappa positive at K/L <1.65 | 12 (16.0) |
| Kappa negative at K/L >1.65 | 45 (63.3) |
| Lambda negative at K/L <0.26 | 6 (16.2) |
| Lambda positive at K/L >0.26 | 28 (59.6) |

FLC = free light chain; K/L = kappa/lambda; UIFE = urine immunofixation electrophoresis.

The described method of urine immunofixation, FLC-UIFE, was significantly more sensitive than the conventional method for detecting monoclonal light chains in urine. The FLC-UIFE method promotes a better utilization of resources and provides a more sensitive detection of monoclonal light chains in urine. These assays were sensitive enough to serve as a marker of residual/minimal residual disease in multiple myeloma. Serum protein electrophoresis, together with serum immunofixation electrophoreses and the new urine immunofixation electrophoreses, FLC-UIFE, obviate the need for serum free light chain assay in diagnosing monoclonal gammopathies.

Serum free light chains (SFLC) are elevated in patients with inflammation and/or chronic renal failure. More than half of the patients with polyclonal hypergammaglobulinemia display abnormal kappa/lambda ratio, almost always a kappa dominant ratio. The development of oligoclonal pattern in patients receiving stem cell transplants renders the use of kappa/lambda ratio useless due to the dominance of kappa light chain associated clones (see Singh, J. Appl. Lab. Med. 5 (2020) 1358-1371.).

Examination of urine is an underutilized test, despite being a non-invasive means of collecting specimens that are critical in diagnosis and monitoring of monoclonal gammopathies. Specifically, (a) ascertainment of complete response to treatment of MM requires absence of monoclonal immunoglobulins in serum and urine; (b) Urine displays monoclonal light chains in all cases of light chain myeloma; (c) UIFE with antisera specific for free light chains (FLC-UIFE) has the potential to improve the detection of malignant monoclonal light chain in patients status-post stem cell transplantation; (d) Results of FLC-modified SIFE and the data support the use of FLC-UIFE for detection of residual/minimal residual disease.

FLC-modified SIFE has been shown to be effective in detecting monoclonal light chains in serum, with a better sensitivity than conventional SIFE and MASS-FIX MALDI. A similar approach has been applied to examination of urine to test for improvement in sensitivity of detection of monoclonal light chains. Monoclonal immunoglobulins are present in serum and/or urine in virtually all cases of neoplastic monoclonal gammopathies with the possible exception the non-secretory MM. Monoclonal immunoglobulins are also preset in body fluids in multiple other disorders, benign and malignant. While the detection of monoclonal immunoglobulins is not diagnostic of MM, evidence of monoclonal proliferation of plasma cells is essential for the diagnosis of MM. Monoclonal intact immunoglobulins and/or monoclonal light chains serve as markers of monoclonal proliferation of plasma cells.

Examination of serum levels of free light chains is recommended by IMWG as well as by an advisory panel of the College of American Pathologists (CAP). It is generally accepted that SFCLA is not a valid test for establishing monoclonality of immunoglobulins or light chains. Quantification of SFLC is essential for establishing a diagnosis of LCPMM and identifying myeloma defining condition based of the concentration of involved light chain and the ratio of involved to uninvolved light chain concentration. Quantification of SFLC is also useful in prognostication and monitoring the course of disease in light chain MM. Despite this understanding it has been proposed that SFLCA can replace urine examination. It has also been documented that an abnormal kappa/lambda ratio is not diagnostic of monoclonality and a normal ratio does not exclude monoclonal gammopathy, let alone MM. A small number of lambda chain associated MM, i.e., IgG lambda and IgA lambda, do not produce/secrete enough free monoclonal lambda light chains to render the kappa/lambda ratio abnormal. The requirement for normal kappa/lambda ratio as a condition for stringent complete response has also been challenged due to high incidence of false positive results in patient status-post stem cell transplantation. In fact, as supported by the data in this communication, it could be posited that FLC-UIFE is more suited for diagnosing monoclonal gammopathy than SFLCA.

Unstructured observations suggested that the only information added by UIFE was the detection of monoclonal light chains in urine. The monoclonal heavy chains and intact monoclonal immunoglobulins are readily detectable in SPEP/SIFE. Detection of monoclonal light chains in urine is important to document and address in treatment due to the known nephrotoxic nature of monoclonal light chains, aka Bence Jones proteins. A systematic examination of UIFE results in 1738 urine specimens validated the impression that the only meaningful information gleaned from UIFE is the documentation of MFLCs. Thus, we propose that conventional UIFE testing for gamma, alpha, and mu chains could be eliminated because it does not provide any value-added information. A separate electrophoresis for UPEP could also be eliminated because conventional UIFE includes a lane for TPr. Therefore, it is contemplated that UIFE is revised from 6 lanes of total protein, anti-gamma, anti-alpha, anti-mu, anti-kappa, and antilambda to 3 lanes for total proteins and kappa and lambda FLCs. It is also contemplated that the increased sensitivity of the method allows for detection of residual disease that would be missed by conventional UIFE. Therefore, it is contemplated that the described methods facilitate a test for MRD in MM patients.

The refinement of UIFE examined in this report strongly favors replacing the conventional antikappa and antilambda sera in UIFE with antisera specific to kappa and lambda FLCs. The 18% additional cases of MLCs detected by FLC-UIFE were in consonance with the expectation from clinical data presented in Table 9. As noted in the FLC-modified SIFE, the additional MLCs detected by the FLC immunofixation electrophoresis is partly due to the detection of MFLCs that have electrophoretic mobility similar to the coexisting intact monoclonal immunoglobulins and are thus not detectable by conventional UIFE. An additional factor in the improved detection rate is likely to be greater sensitivity of FLC-UIFE, as evidenced by the detection of MLCs in urine with a serum concentration of 0.48 mg/L, compared with the corresponding level of 1.19 mg/L detected by conventional UIFE for kappa LCs when both methods were used in parallel.

The markedly different rates of positive findings on UIFE at the 3 institutions reflect the wide variation in utilization of UIFE. At institution A, the neurology and nephrology services are prominent users of UIFE, whereas the oncology service does not routinely order UIFE due to clinician preference. At institution B, the hematology/oncology service is the dominant user of UIFE. At institution C, the hematology/oncology service was the largest user, with sizeable contributions from rheumatology, neurology, and nephrology service. Despite the markedly different rates of finding MLCs in urine, the low SFLC levels and the low ratio of involved to uninvolved SFLC concentrations associated with detection of MLCs in urine are remarkably similar at the 3 institutions. Moreover, the negative results at high levels of SFLC and the ratio of involved to uninvolved SFLC concentrations also show similar trends at the 3 institutions. These findings highlight the previously documented observation of poor correlation between SFLC levels and presence of monoclonal light chains.

The lack of detection of a monoclonal kappa light chain in urine by FLC-UIFE in a patient with IgM Kappa monoclonal immunoglobulin in serum likely reflects the lack of appropriate epitopes in the immunogens used for production of antibodies A similar negative result was also document by Binding Site that was corrected by changes to the immunogen. (Badwell A R. Serum Free Light Chain Analysis Plus Hevylite. seventh ed. ISBN 780-0-9932196-0-3.)

A monoclonal kappa light chain was detected by conventional UIFE at the initial examination. Testing by conventional and FLC-UIFE was repeated to ensure that the lack of reactivity with FLC-UIFE was not due to deterioration of specimen on storage.

As an alternative to the recommendations by IMWG and CAP panel, diagnostic work up for monoclonal gammopathy should include SPEP, SIFE and FLC-UIFE only. SFLCA may be carried out to diagnose LCPMM and for monitoring of light chain MM. The criteria for myeloma defining condition based on light chain concentration have been challenged as the criteria, as presented, are not light chain specific despite documentation of marked excess of free kappa chain than free lambda chains.

FLC-UIFE could serve as a marker of minimal residual disease (MRD), especially in patients with light chain MM and LCPMM.

The only useful information added by UIFE, over SPEP and SIFE, is detection of MFLCs. All instances in which intact monoclonal immunoglobulins could be detected by UIFE were in patients with positive SIFE results for the cognate immunoglobulin. UIFE with antisera to free LCs detects 18% more instances of MLCs in urine than conventional UIFE.

Based on these findings, the following diagnostic algorithms are contemplated for screening for monoclonal gammopathy:
Screening for monoclonal gammopathy should consist of SPEP, SIFE, and FLC-UIFE only. SFLCA does not add value due to the large number of false-negative and false-positive results it yields.
FLC-UIFE should be performed in the work-up for diagnosis of monoclonal gammopathy and for detection of residual disease in patients treated for MM.
FLC-UIFE should be performed in the aforementioned circumstance irrespective of the serum level of FLCs and the ratio of I/U LC concentration.
FLC-UIFE should be performed in all specimens, notwithstanding TPr in the normal range.
FLC-UIFE promotes better utilization of resources and provides a more-sensitive method for detection of MLCs in urine.
The role of FLC-UIFE as a test for MRD should be explored, as has been suggested for FLC-modified SIFE.

### REFERENCES

R. A. Kyle, D. R. Larson, T. M. Therneau, A. Dispenzieri, S. Kumar, J. R. Cerhan, et al., Long-term follow-up of monoclonal gammopathy of undetermined significance, N. Engl. J. Med. 378 (2018) 241-249.
A. Lakshman, S. V. Rajkumar, F. K. Buadi, M. Binder, M. A. Gertz, M. Q. Lacy, et al., Risk stratification of smoldering multiple myeloma incorporating revised IMWG diagnostic criteria, Blood Cancer J. 12 (8) (2018) 59-69.
A. Palumbo, K. Anderson, N Multiple myeloma, N. Engl. J. Med. 364 (2011) 1046-1060.
G. Singh, Serum and urine protein electrophoresis and serum-free light chain assays in the diagnosis and monitoring of monoclonal gammopathies, J. Appl. Lab. Med. 5 (2020) 1358-1371.
R. Fonseca, M. Gonzalez-Velez, Treatment of smoldering multiple myeloma: expectant observation should still Be the standard, Am. Soc. Clin. Oncol. Educ. Book 40 (2020) 1-7.
S. Lonial, S. Jacobus, R. Fonseca, M. Weiss, S. Kumar, R. Z. Orlowski, et al., Randomized trial of lenalidomide versus observation in smoldering multiple myeloma, J. Clin. Oncol. 38 (2020) 1126-1137.
E. B. Kim, A. J. Yee, N. Raje, Treatment of smoldering multiple myeloma: ready for prime time? Cancers 12 (2020) 1223-1240.
D. Kazandjian, Multiple myeloma epidemiology and survival: a unique malignancy, Semin. Oncol. 43 (2016) 676-681.
B. Dhakal, A. Szabo, S. Chhabra, M. Hamadani, A. D'Souza, S. Z. Usmani, et al., Autologous transplantation for newly diagnosed multiple myeloma in the era of novel agent induction: a systematic review and meta-analysis, JAMA Oncol. 4 (2018) 343-350.
M. Attal, V. Lauwers-Cances, C. Hulin, X. Leleu, D. Caillot, M. Escoffre, et al., Lenalidomide, bortezomib, and dexamethasone with transplantation for myeloma, N. Engl. J. Med. 376 (2017) 1311-1320.
Y. Jin, N. M. Savage, R. Bollag, H. Xu, G. Singh, Light chain multiple myeloma: high serum free light chain concentrations portend renal damage and poorer survival, jfab090, J. Appl. Lab. Med. (2021 Sep. 1), https://doi.org/10.1093/jalm/jfab090. Online ahead of print.
P. Sonneveld, H. Avet-Loiseau, S. Lonial, S. Usmani, D. Siegel, K. C. Anderson, et al., Treatment of multiple myeloma with high-risk cytogenetics: a consensus of the International Myeloma Working Group, Blood 127 (2016) 2955-2962.
G. Singh, H. Xu, Light chain predominant intact immunoglobulin monoclonal gammopathy disorders: shorter survival in light chain predominant multiple myelomas, lmaa057, Lab. Med. (2020 Nov. 12), https://doi.org/10.1093/labmed/lmaa057. Online ahead of print.
Singh G, Savage N, Jillella A, Bollag R. Light chain-predominant multiple myeloma subgroup; Impaired renal function correlates with decreased survival in this subgroup. Lab Med. 2021; 53:145-148 lmab054, https://doi.org/10.1093/labmed/lmab054.
S. Manohar, S. H. Nasr, N. Leung, Light chain cast nephropathy: practical considerations in the management of myeloma kidney-what we know and what the future may hold, Curr Hematol. Malig. Rep. 13 (2018) 220-226.
D. F. Keren, L. Schroeder, Challenges of measuring monoclonal proteins in serum, Clin. Chem. Lab. Med. 54 (2016) 947-961.
N. Omar, K. Madwani, P. Moideen, D. Manthei, D. Keren, G. Singh, Accurate quantification of monoclonal immunoglobulins migrating in the beta region on protein electrophoresis, Lab. Med (2021 Aug. 13) lmab055. doi: https://doi.org/10.1093/labmed/lmab055. Online ahead of print.
Badwell A R. Serum Free Light Chain Analysis Plus Hevylite. seventh ed. ISBN 780-0-9932196-0-3.
W. S. Lee, G. Singh, Serum free light chain assay in monoclonal gammopathic manifestations, Lab. Med. 50 (2019) 381-389.
G. Singh, Concentrations of serum free light chains in kappa and lambda lesions in light-chain myelomas, Lab. Med. 50 (2) (2019) 189-193.
W. S. Lee, G. Singh, Serum free light chains in neoplastic monoclonal gammopathies: relative under-detection of lambda dominant kappa/lambda ratio, and underproduction of free lambda light chains, as compared to kappa light chains, in patients with neoplastic monoclonal gammopathies, J. Clin. Med. Res. (2018) 562-569.
G. Singh, Serum free light chain assay and κ/λ ratio performance in patients without monoclonal gammopathies: HighFalse-PositiveRate, Am. J. Clin. Pathol. 146 (2016) 207-214.
G. Singh, Serum free light chain assay and κ/λ ratio: performance in patients with monoclonal gammopathy-high false negative rate for κ/λ ratio, J. Clin. Med. Res. 9 (2017) 46-57.
G. Singh, Oligoclonal pattern/abnormal protein bands in post-treatment plasma cell myeloma patients: implications for protein electrophoresis and serum free light chain assay results, J. Clin. Med. Res. 9 (2017) 671-679.
L. Sepiashvili, M. C. Kohlhagen, M. R. Snyder, M. A. V. Willrich, J. R. Mills, A. Dispenzieri, D. L. Murray, Direct detection of monoclonal free light chains in serum by use of immunoenrichment-coupled MALDI-TOF mass spectrometry, Clin. Chem. 65 (2019) 1015-1022.
Wilhite D, Arfa A, Cotter T, Savage N M, Bollag R J, Singh G. Multiple Myeloma: Detection of free monoclonal light chains by modified immunofixation electrophoresis with antisera against free light chain. Pract Lab Med 2021; 27:e00256
J. R. Mills, M. C. Kohlhagen, S. Dasari, P. M. Vanderboom, R. A. Kyle, J. A. Katzmann, et al., Comprehensive assessment of M-proteins using nanobody enrichmentcoupled to MALDI-TOF mass spectrometry, Clin. Chem. 62 (2016) 1334-1344.
M. Zajec, P. Langerhorst, M. M. VanDuijn, J. Gloerich, H. Russcher, A. J. van Gool, et al., Mass spectrometry for identification, monitoring, and minimal residual disease detection of M-proteins, Clin. Chem. 66 (2020) 421-433.
P. Milani, D. L. Murray, D. R. Barnidge, M. C. Kohlhagen, J. R. Mills, G. Merlini, et al., The utility of MASS-FIX to detect and monitor monoclonal proteins in the clinic, Am. J. Hematol. 92 (2017) 772-779.
G. Singh, Bollag R quantification by ultrafiltration and immunofixation electrophoresis testing for monoclonal serum free light chains, Lab. Med. 51 (2020) 592-600.
O. V. Nwogbo, Y. Jin, T. Sliker, D. Wilhite, G. Singh, Analysis of multiple bands on serum protein immunofixation electrophoresis: challenge in interpretation of clonality in a patient with light chain-predominant multiple myeloma, lmab001, Lab. Med. (2021 Mar. 8), https://doi.org/10.1093/labmed/lmab001. Online ahead of print.
L. Mikkilineni, J. N. Kochenderfer, CAR T cell therapies for patients with multiple myeloma, Nat. Rev. Clin. Oncol. 18 (2021) 71-84.

Unless defined otherwise, all technical and scientific terms used herein have the same meanings as commonly understood by one of skill in the art to which the disclosure belongs. Publications cited herein and the materials for which they are cited are specifically incorporated by reference in their entireties.

Those skilled in the art will recognize or be able to ascertain using no more than routine experimentation, many equivalents to the specific embodiments of the disclosure. Such equivalents are intended to be encompassed by the following claims.

We claim:

1. A method of identifying the presence of serum free monoclonal light chains in an undiluted serum sample, or other body fluid or extract of cells or tissue from a subject by immunofixation electrophoresis, comprising
(i) depositing at least one aliquot portion of the undiluted serum sample on a deposit area of an electrophoretic gel plate having one anodic side and one cathodic side,
wherein the sample deposit area is at a position of the gel plate allowing electrophoretic migration of negatively charged/acidic proteins within the deposited undiluted serum sample towards the anodic side of the gel plate and migration of positively charged/basic proteins towards the cathodic end of the gel plate;
(ii) electrophoresing the gel plate to provide an undiluted serum protein separation profile;
(iii) contacting the electrophoresed gel with a solution comprising at least one capture antibody,
wherein the capture antibody has specificity for free immunoglobulin light chain proteins or fragments thereof,
wherein the contacting is under conditions that permit the formation of precipitate and/or detectable immunocomplexes between the capture antibody and free immunoglobulin light chain proteins or fragments thereof within the protein separation profile;
(iv) removing unbound capture antibody,
wherein the removing comprises blotting the solution comprising at least one capture antibody by contacting the gel with blotting paper;
(v) contacting the gel with a wash solution for at least one minute, preferably three minutes, then removing the wash solution;
(vi) optionally, repeating step (v) from one to ten times, inclusive; and
(vii) optionally, staining and/or quantitating the immunocomplexes formed in step (iii).

2. The method of claim 1, wherein the concentration of free monoclonal immunoglobulin light chain in the undiluted sample is 1.75 mg/L or more.

3. The method of claim 1, wherein contacting the gel with a wash solution comprises:
(a) contacting the gel with a saline wash solution;
(b) incubating the gel in the wash solution for 3 min;
(c) contacting the gel with blotting paper to remove the wash solution;
(d) repeating steps (a-c) twice or more times;
(e) contacting the gel with blotting paper filter by overlaying the gel with the paper and saturating the paper with saline wash solution;
(f) incubating the gel in the wash solution for 3 min;
(g) removing the filter paper and contacting the gel with more blotting paper filters to remove the wash solution; and
(h) repeating steps (d-f) two or more times.

4. The method of claim 1, wherein at least one aliquot portion of the undiluted sample is deposited on the gel plate as a reference which is not submitted to step (iii) but is instead contacted with a fixative solution rather than with capture antibody(ies),
wherein steps (i), (ii), (iv), (v), and optionally (vi) and/or (vii) remain the same.

5. The method of claim 1, wherein the sample is a serum sample from a human subject.

6. The method of claim 4, further comprising one or more steps of (viii) analyzing and/or interpreting the immunofixation electrophoresis results and/or concluding about the health status of the subject; and
(ix) optionally, treating the subject for a disease when the sample contains monoclonal immunoglobulins and meets one or more additional criteria for the diagnosis of one or more disease or disorder associated with free monoclonal light chains.

7. The method of claim 6, wherein the disease or disorder associated with free monoclonal light chains is selected from the group consisting of monoclonal gammopathy of undetermined significance (MGUS), asymptomatic or smoldering multiple myeloma (SMM), multiple/plasma cell myeloma (MM), HIV/AIDS, Chronic lymphocytic leukemia, Non-Hodgkin Lymphoma, particularly Splenic marginal zone lymphoma and Lymphoplasmacytic lymphoma, Hepatitis C, Connective tissue disease such as lupus, Immunosuppression following organ transplantation, Waldenstrom macroglobulinemia, Guillain-Barre syndrome, polyneuropathy, amyloidosis and Tempi syndrome.

8. The method of claim 7, wherein the disease or disorder associated with free monoclonal light chains is a conventional MM, light-chain-predominant multiple/plasma cell myeloma (LCPMM) or light chain myeloma/LCMM.

9. The method of claim 5, wherein the subject has previously been treated for a disease or disorder associated with free monoclonal light chains selected from the group consisting of monoclonal gammopathy of undetermined significance (MGUS), asymptomatic or smoldering multiple myeloma (SMM), multiple/plasma cell myeloma (MM), HIV/AIDS, Chronic lymphocytic leukemia, Non-Hodgkin Lymphoma, particularly Splenic marginal zone lymphoma and Lymphoplasmacytic lymphoma, Hepatitis C, Connective tissue disease such as lupus, Immunosuppression following organ transplantation, Waldenstrom macroglobulinemia, Guillain-Barre syndrome, polyneuropathy, amyloidosis or Tempi syndrome.

10. The method of claim 9, wherein the subject has received, or is receiving treatment for a Neoplastic monoclonal gammopathy (NMG),
optionally, wherein the treatment comprises chemotherapy, immunotherapy, corticosteroids, targeted therapy, radiation therapy, proteasome inhibition, monoclonal antibodies against CD38 and/or SLAM7, antibody-drug conjugate therapy, nuclear export inhibition, bisphosphonate treatment for bone disease, CAR T cell therapy, autologous stem cell transplantation (ASCT), or a combination thereof.

11. A method of identifying the presence of free monoclonal light chains in a urine sample from a subject by immunofixation electrophoresis, comprising
(i) concentrating a urine sample comprising proteins to provide a concentrated urine sample,
wherein the concentrating is effective to reduce the volume of the sample from about 5-fold to about 200-fold, inclusive;
(ii) depositing at least one aliquot portion of the concentrated urine sample on a deposit area of an electrophoretic gel plate having one anodic side and one cathodic side,
wherein the sample deposit area is at a position of the gel plate allowing electrophoretic migration of negatively charged/acidic proteins within the deposited concentrated urine sample towards the anodic side of the gel plate and migration of positively charged/basic proteins towards the cathodic end of the gel plate;
(iii) electrophoresing the gel plate to provide a protein separation profile of the concentrated urine sample;
(iv) contacting the electrophoresed gel with a solution comprising at least one capture antibody,
wherein the capture antibody has specificity for free immunoglobulin light chain proteins or fragments thereof, and
wherein the contacting is under conditions that permit formation of precipitate and/or detectable immunocomplexes between the capture antibody and free immunoglobulin light chain proteins or fragments thereof within the protein separation profile;
(v) removing unbound capture antibody,
wherein the removing comprises blotting the solution comprising at least one capture antibody by contacting the gel with blotting paper;
(vi) contacting the gel with a wash solution for at least one minute, preferably three minutes, then removing the wash solution;
(vii) optionally, repeating step (v) from one to ten times, inclusive; and
(viii) optionally, staining and/or quantitating the immunocomplexes formed in step (iv).

12. The method of claim 11, wherein contacting the gel with a wash solution in step (vi) comprises one or more steps of:
(a) contacting the gel with a saline wash solution;
(b) incubating the gel in the wash solution for 3 min;
(c) contacting the gel with blotting paper to remove the wash solution;
(d) repeating steps (a-c) twice or more times;
(e) contacting the gel with blotting paper filter by overlaying the gel with the paper and saturating the paper with saline wash solution;
(f) incubating the gel in the wash solution for 3 min;
(g) removing the filter paper and contacting the gel with more blotting paper filters to remove the wash solution; and
(h) repeating steps (d-f) two or more times.

13. The method of claim 11, wherein the staining and/or quantitating the immunocomplexes in step (iv) comprises drying the gel and staining the gel with a dye suitable for quantitation and visual examination.

14. The method of claim 11, wherein at least one aliquot portion of the undiluted sample is deposited on the gel plate as a reference which is not submitted to step (iv) but is instead contacted with a fixative solution rather than with capture antibody(ies),
wherein steps (i), (ii), (iii), (v), and optionally steps (vi), (vii), and/or (viii) remain the same.

15. The method of claim 11, further comprising one or more steps of
(ix) analyzing and/or interpreting the immunofixation electrophoresis results and/or concluding about the health status of the subject; and
(x) optionally, treating the subject for a disease when the sample contains monoclonal immunoglobulins and meets one or more additional criteria for the diagnosis of one or more disease or disorder associated with free monoclonal light chains.

16. The method of claim 15, wherein the disease or disorder associated with free monoclonal light chains is selected from the group consisting of monoclonal gammopathy of undetermined significance (MGUS), asymptomatic or smoldering multiple myeloma (SMM), multiple/plasma cell myeloma (MM), HIV/AIDS, Chronic lymphocytic leukemia, Non-Hodgkin Lymphoma, particularly Splenic marginal zone lymphoma and Lymphoplasmacytic lymphoma, Hepatitis C, Connective tissue disease such as lupus, Immunosuppression following organ transplantation, Waldenstrom macroglobulinemia, Guillain-Barre syndrome, polyneuropathy, amyloidosis and Tempi syndrome.

17. The method of claim 16, wherein the disease or disorder associated with free monoclonal light chains is a conventional MM, light-chain-predominant multiple/plasma cell myeloma (LCPMM) or light chain myeloma/LCMM.

18. The method of claim 11 wherein the subject has previously been treated for a disease or disorder associated with free monoclonal light chains selected from the group consisting of monoclonal gammopathy of undetermined significance (MGUS), asymptomatic or smoldering multiple myeloma (SMM), multiple/plasma cell myeloma (MM), HIV/AIDS, Chronic lymphocytic leukemia, Non-Hodgkin Lymphoma, particularly Splenic marginal zone lymphoma and Lymphoplasmacytic lymphoma, Hepatitis C, Connective tissue disease, lupus erythematosus, Immunosuppression following organ transplantation, Waldenstrom macroglobulinemia, Guillain-Barre syndrome, polyneuropathy, amyloidosis and Tempi syndrome.

19. The method of claim 18, wherein the subject has received, or is receiving treatment for a Neoplastic monoclonal gammopathy (NMG),
optionally, wherein the treatment comprises one or more of chemotherapy, immunotherapy, corticosteroids, targeted therapy, radiation therapy, proteasome inhibition, monoclonal antibodies against CD38 and/or SLAM7, antibody-drug conjugate therapy, nuclear export inhibition, bisphosphonate treatment for bone disease, CAR T cell therapy, autologous stem cell transplantation (ASCT), or a combination thereof.

20. A kit suitable for carrying out a method according to claim 1, comprising
(i) instructions according to the method of claim 1; and
one or more of
(ii) capture antibody(ies) specific for free immunoglobulin light chain;
(iii) electrophoretic gels;
(iv) negative control samples comprising no free monoclonal immunoglobulin light chain;
(v) blotting paper;
(vi) wash solution;
(vii) fixative solution;
(viii) Gel stain;
(ix) positive control samples comprising a known amount and type of free immunoglobulin light chain; and
(x) apparatus for obtaining a biological sample from a subject.
